(19) United States
(12) Patent Application Publication
(10) Pub. No.: US 2025/0342906 A1
(43) Pub. Date: Nov. 6, 2025

(54) EPITOPE AND IMMUNOGEN DESIGN FOR DEVELOPMENT OF VACCINES, DIAGNOSTICS, AND IMMUNOTHERAPEUTICS

(71) Applicant: Iowa State University Research Foundation, Inc., Ames, IA (US)

(72) Inventors: Ratul Chowdhury, Ames, IA (US); Supantha Dey, Ames, IA (US); Michael Roof, Ames, IA (US)

(21) Appl. No.: 19/183,263

(22) Filed: Apr. 18, 2025

Related U.S. Application Data
(60) Provisional application No. 63/635,940, filed on Apr. 18, 2024.

(51) Int. Cl. G16B 15/30; A61K 39/215; C07K 14/005; C07K 16/10; C12N 7/00; G16B 15/20; G16B 50/30

(52) U.S. Cl. CPC G16B 15/30; A61K 39/215; C07K 14/005; C07K 16/10; C12N 7/00; G16B 15/20; G16B 50/30; C12N 2770/10022; C12N 2770/10034; C12N 2770/20022; C12N 2770/20034

(57) ABSTRACT

Workflows for the efficient identification of viral epitopes and/or host paratopes are provided. The workflows leverage artificial intelligence to quickly and reliably identify candidate epitopes for immunogen development thereby reducing the lead time of vaccine development. Immunogenic compositions for use in the treatment and/or prevention of porcine reproductive and respiratory syndrome virus (PRRSV) and Infectious Bronchitis Virus (IBV) are also provided, as are antibodies or antigen binding fragments.

### CROSS REFERENCE TO RELATED APPLICATIONS

This application claims priority under 35 U.S.C. § 119 to provisional patent application U.S. Ser. No. 63/635,940, filed Apr. 18, 2024. The provisional patent application is herein incorporated by reference in its entirety, including without limitation, the specification, claims, and abstract, as well as any figures, tables, appendices, or drawings thereof.

### REFERENCE TO SEQUENCE LISTING SUBMITTED ELECTRONICALLY

The instant application contains a Sequence Listing which has been submitted electronically in XML format and is herein incorporated by reference in its entirety. Said XML copy, created on Apr. 15, 2025, is named “P14770US01_SequenceListing.xml” and is 56,386 bytes in size.

### TECHNICAL FIELD

The present disclosure relates generally to methods utilizing artificial intelligence for the identification of vaccine epitopes, immunogen design, and therapeutic, diagnostic, and vaccine development.

### BACKGROUND

Viruses can have devastating public health and food supply consequences. Despite accessibility of vaccines for some viruses, obstacles persist because vaccines do not always generate comprehensive protection. This is primarily due to the variability of viral strains and the intricate nature of the interplay between the virus and the immune responses of the host. For many viruses, current strategies for controlling infection have been mostly insufficient.

Viruses can develop mechanisms to escape the host immune response using various processes. The diversity of a viral strain is significantly influenced by high-frequency mutation and recombination between different lineages/sub-lineages. Furthermore, mutations can influence the rate of viral reinfection and the duration of vaccine effectiveness. Therefore, the key to developing optimal vaccines and therapeutics depends on anticipating viral variants that can elude immune detection with enough lead time. For diverse viruses or viruses with a high frequency of mutation, prior art methods of identifying viral epitope proteins and designing immunogens targeting these proteins are limited.

Therefore, there is a need in the art to develop methods that can reliably and efficiently predict viral escape and quickly identify candidate proteins for vaccine synthesis.

### SUMMARY

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage to address previous challenges of reliably, efficiently, and quickly identifying candidate proteins for vaccine synthesis and potential immunotherapies.

It is a further object, feature, and/or advantage to provide novel porcine reproductive and respiratory syndrome virus (PRRSV) vaccinations, immunogenic compositions, antigens, and antibodies.

According to some aspects of the present disclosure, a computerized method for identifying viral epitopes and/or paratopes is provided. In some embodiments, the method comprises identifying one or more viral proteins; identifying one or more host receptor proteins; performing subcellular localization of the one or more viral proteins and the one or more host receptor proteins to identify one or more transmembrane viral proteins and one or more transmembrane host receptor proteins; predicting the three dimensional structure of the one or more transmembrane viral proteins and the one or more transmembrane host receptor proteins; predicting protein-protein docking poses of the of the one or more transmembrane viral proteins to the one or more transmembrane host receptor proteins; identifying one or more viral epitopes and/or one or more host receptor paratopes based on the predicted protein-protein docking poses, wherein at least one step is performed using artificial intelligence.

In some embodiments, the methods provided herein further comprise generating a database of identified viral epitopes.

According to some aspects of the present disclosure, a computerized method for designing viral antibodies is provided. In some embodiments, the method comprises constructing an immunogen comprising at least one viral epitope from a database of identified viral epitopes and using artificial intelligence to design an antibody reactive against the immunogen.

According to some aspects of the present disclosure an immunogenic composition is provided. In some embodiments, the immunogenic composition comprises an antigenic fragment of a porcine reproductive and respiratory syndrome virus (PRRSV) protein, wherein the protein is E envelope protein, GP2, GP3, GP4, GP5, membrane protein M, nsp3, nsp5, and/or ORF5a; and a pharmaceutically acceptable carrier.

According to some aspects of the present disclosure, an antibody or an antigen binding fragment thereof capable of binding to an antigenic fragment of a PRRSV protein is provided. In some embodiments, the PRRSV protein is E envelope protein, GP2, GP3, GP4, GP5, membrane protein M, nsp3, nsp5, and/or ORF5a.

According to some aspects of the present disclosure, a multi-epitope vaccine (MEV) is provided. In some embodiments, the MEV comprises at least two antigenic fragments of an Infectious Bronchitis Virus (IBV) protein, wherein the antigenic fragments comprise at least one B-cell epitope and at least one T-cell epitope.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIG. 1 shows a general workflow for a method of identifying viral epitopes and/or paratopes as described in some embodiments of the present disclosure.

FIG. 2 shows a workflow for a method of identifying porcine reproductive and respiratory syndrome virus (PRRSV) epitopes and/or paratopes as described in Example 1.

FIG. 3 shows a bar graph illustrating the number of unique PRRSV epitopes interacting with swine in each complex.

FIG. 4 shows the computational Energy scores of all the PRRSV docking poses predicted by ClusPro 2.0, GRAMM Dock, and HADDOCK.

FIG. 5 shows the top energy complexes from the interacting PRRSV-swine proteins of interest.

FIG. 6 shows a bar graph illustrating the top immunogens with a cut of RMSD 2.0 Å after relaxation.

FIGS. 7A-C: (7A) Schematic of the process for identifying MHC-I, MHC-II, and B cell epitopes for N and spike proteins from the IBV genome. (7B) Analyzing the conservancy of predicted epitopes across 56 IBV genomes. The analysis highlights unique epitopes within the most conserved regions of the N and spike proteins, identifying potential targets for broad-spectrum vaccine development. (7C) Allergenicity, toxicity and antigenicity analysis for the final selection of the epitopes.

FIGS. 8A-B shows docked complexes of MEVs with chicken TLR4 (8A) SmallTope (right) and (8B) BigTope (right) bound to chTLR4 (left).

FIG. 9 shows a comprehensive evaluation process for MEV candidates designed using RFdiffusion and ProteinMPNN. The workflow integrates binding affinity assessment, secondary structure comparison, and sequence alignment analysis to ensure optimal structural integrity and immunogenic potential.

An artisan of ordinary skill in the art need not view, within isolated figure(s), the near infinite distinct combinations of features described in the following detailed description to facilitate an understanding of the present disclosure.

### DETAILED DESCRIPTION

So that the present disclosure may be more readily understood, certain terms are first defined. Unless defined otherwise, all technical and scientific terms used herein have the same meaning as commonly understood by one of ordinary skill in the art to which embodiments of the disclosure pertain. The definitions are provided to aid in describing particular embodiments and are not intended to limit the claimed disclosure. Many methods and materials similar, modified, or equivalent to those described herein can be used in the practice of the embodiments without undue experimentation, but the preferred materials and methods are described herein. In describing and claiming the embodiments, the following terminology will be used in accordance with the definitions set out below.

It is to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form. Numeric ranges recited within the specification are inclusive of the numbers within the defined range. Throughout this disclosure, various aspects are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range (e.g., 1 to 5 includes 1, 1.5, 2, 2.75, 3, 3.80, 4, and 5).

As used herein, the term “and/or”, e.g., “X and/or Y” shall be understood to mean either “X and Y” or “X or Y” and shall be taken to provide explicit support for both meanings or for either meaning, e.g., A and/or B includes the options i) A, ii) B or iii) A and B.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

The term “about,” as used herein, refers to variation in the numerical quantity that can occur, for example, through typical measuring and liquid handling procedures used for making concentrates or use solutions in the real world; through inadvertent error in these procedures; through differences in the manufacture, source, or purity of the ingredients used to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

“Antibodies” refers to polyclonal and monoclonal antibodies, chimeric, and single chain antibodies, as well as Fab fragments, including the products of a Fab or other immunoglobulin expression library. With respect to antibodies, the term, “immunologically specific” refers to antibodies that bind to one or more epitopes of a protein of interest, but which do not substantially recognize and bind other molecules in a sample containing a mixed population of antigenic biological molecules.

An “attenuated” virus as used herein refers to a virus which is capable of infecting and/or replicating in a susceptible host but is non-pathogenic or less-pathogenic to the susceptible host. For example, the attenuated virus may cause no observable/detectable clinical manifestations, or less clinical manifestations, or less severe clinical manifestations, or exhibit a reduction in viral replication efficiency and/or infectivity, as compared with the related field isolated/wild-type strains.

An “immunogenic or immunological composition” refers to a composition of matter that comprises at least one antigen, which elicits an immunological response in the host of a cellular and/or antibody-mediated immune response to the composition or vaccine of interest. Usually, an “immune response” or “immunological response” includes but is not limited to one or more of the following effects: the production or activation of antibodies, B cells, helper T cells, suppressor T cells, and/or cytotoxic T cells and/or gamma-delta T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction or lack of clinical signs normally displayed by an infected host, a quicker recovery time and/or a lowered duration or viral load in the tissues or body fluids or excretions of the infected host compared to a healthy control. Preferably said reduction in symptoms is statistically significant when compared to a control. In some embodiments, the immunogenic compositions may be used as a “vaccine”. The term “vaccine”, as used herein, refers to an antigenic preparation used to produce immunity to a disease, in order to prevent or ameliorate the effects of infection. Vaccines are typically prepared using a combination of an immunologically effective amount of an immunogen together with an adjuvant effective for enhancing the immune response of the vaccinated subject against the immunogen.

The terms “include” and “including” when used in reference to a list of materials refer to but are not limited to the materials so listed.

As used herein, “a pharmaceutically acceptable carrier” or “pharmaceutical carrier” includes any and all excipients, solvents, growth media, dispersion media, coatings, adjuvants, stabilizing agents, diluents, preservatives, inactivating agents, antimicrobial, antibacterial and antifungal agents, isotonic agents, adsorption delaying agents, and the like. Such ingredients also include those that are safe and appropriate for use in veterinary applications. Pharmaceutically acceptable carriers are typically non-toxic, inert, solid or liquid carriers.

The term “subject” as used herein refers to any living being that would benefit from the compositions and methods described herein. For example, the subject may be an animal, including a human, avian, bovine, canine, equine, feline, hircine, lupine, murine, ovine, and porcine animal. Subjects may also be domesticated animals such as cats, dogs, rabbits, guinea pigs, ferrets, hamsters, mice, gerbils, horses, cows, goats, sheep, donkeys, pigs, and the like. Avian animals includes poultry animals, such as chickens, turkeys, ducks, geese, guinea fowl, pigeons, ostrich, emu, partridge, pheasant, and the like. In certain embodiments, the subject is a human. In certain embodiments, the subject is a pig. In certain embodiments, the subject is an avian animal, such as chickens.

Immunogenic compositions will contain a “therapeutically effective amount” of the active ingredient, that is, an amount capable of eliciting an induction of an immunoprotective response in a subject to which the composition is administered. In the treatment and prevention of viral infections, for example, a “therapeutically effective amount” would preferably be an amount that enhances resistance of the immunized subject to new infection and/or reduces the clinical severity of the disease. Such protection will be demonstrated by either a reduction or lack of symptoms normally displayed by a subject infected with the virus, a quicker recovery time and/or a lowered viral load. Immunogenic compositions can be administered prior to infection, as a preventative measure against viral infection. Alternatively, immunogenic compositions can be administered after the subject has already showed clinical manifestations of infection. Immunogenic compositions given after manifestations of viral infection may be able to attenuate the infection, triggering a superior immune response than the natural infection itself.

The present disclosure provides for reduction of the incidence of and/or severity of clinical symptoms and/or reduction of viral load associated with viral infection. Preferably, the severity and/or incidence of clinical symptoms and/or viral load in subjects receiving the immunogenic composition of the present disclosure are reduced at least 10% in comparison to subjects not receiving such an administration when both groups (subjects receiving and subjects not receiving the composition) are challenged with or exposed to infection by the virus. In some embodiments, the incidence or severity and/or viral load is reduced at least 20%, at least 30%, at least 40%, at least 50%, at least 60%, at least 70%, at least 80%, at least 90%, at least 95%, or 100%, wherein the subjects receiving the composition of the present disclosure exhibit no clinical symptoms or no viral load, or alternatively exhibit clinical symptoms of reduced severity or reduced viral load.

The term “weight percent,” “wt. %,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt. %,” etc.

The methods and compositions may comprise, consist essentially of, or consist of the components and ingredients as well as other ingredients described herein. As used herein, “consisting essentially of” means that the methods and compositions may include additional steps, components or ingredients, but only if the additional steps, components or ingredients do not materially alter the basic and novel characteristics of the claimed methods and compositions.

Aspects and/or embodiments of the present disclosure aim to overcome and/or improve on issues and challenges raised. At least one goal is to leverage artificial intelligence to reliably, efficiently, and quickly identify candidate proteins for vaccine synthesis, including viral vaccine synthesis.

Aspects and/or embodiments of the present disclosure provide a computerized workflow for identifying viral epitopes, immunogens, and/or host paratopes.

### Software, Computer System, and Network Environment

Some embodiments described herein make use of computer algorithms in the form of software instructions executed by a computer processor. In some embodiments, the software instructions include a machine learning module, also referred to herein as artificial intelligence software. As used herein, a machine learning module refers to a computer implemented process (e.g., a software function) that implements one or more specific machine learning algorithms, such as an artificial neural network (ANN), convolutional neural network (CNN), random forest, decision trees, support vector machines, and the like, in order to determine, for a given input, one or more output values. In some embodiments, the input comprises alphanumeric data which can include numbers, words, phrases, or lengthier strings, for example. In some embodiments, the one or more output values comprise values representing numeric values, words, phrases, or other alphanumeric strings. In some embodiments, the one or more output values comprise an identification of one or more response strings (e.g., selected from a database).

For example, a machine learning module may receive as input a textual string (e.g., entered by a human user, for example) and generate various outputs. For example, the machine learning module may automatically analyze the input alphanumeric string(s) to determine output values classifying a content of the text (e.g., an intent).

In some embodiments, machine learning modules implementing machine learning techniques are trained, for example using datasets that include categories of data described herein. Such training may be used to determine various parameters of machine learning algorithms implemented by a machine learning module, such as weights associated with layers in neural networks. In some embodiments, once a machine learning module is trained, e.g., to accomplish a specific task such as identifying certain response strings, values of determined parameters are fixed and the (e.g., unchanging, static) machine learning module is used to process new data (e.g., different from the training data) and accomplish its trained task without further updates to its parameters (e.g., the machine learning module does not receive feedback and/or updates). In some embodiments, machine learning modules may receive feedback, e.g., based on user review of accuracy, and such feedback may be used as additional training data, to dynamically update the machine learning module. In some embodiments, two or more machine learning modules may be combined and implemented as a single module and/or a single software application. In some embodiments, two or more machine learning modules may also be implemented separately, e.g., as separate software applications. A machine learning module may be software and/or hardware. For example, a machine learning module may be implemented entirely as software, or certain functions of a ANN module may be carried out via specialized hardware (e.g., via an application specific integrated circuit (ASIC)).

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, some modules described herein can be separated, combined or incorporated into single or combined modules. Any modules depicted in the figures are not intended to limit the systems described herein to the software architectures shown therein.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein.

While the methods and systems of present disclosure has been particularly shown and described with reference to specific preferred embodiments, it should be understood that various changes in form and detail may be made therein without departing from the spirit and scope of the present disclosure.

### AI/Machine Learning

Many statistical classification techniques are suitable as approaches to perform the classification described herein. Such methods include but are not limited to supervised learning approaches.

Commonly used supervised classifiers include without limitation the neural network (e.g., artificial neural network, multi-layer perceptron), support vector machines, k-nearest neighbors, Gaussian mixture model, Gaussian, naive Bayes, decision tree and radial basis function (RBF) classifiers. Linear classification methods include Fisher's linear discriminant, logistic regression, naive Bayes classifier, perceptron, and support vector machines (SVMs). Other classifiers for use with methods according to the disclosure include quadratic classifiers, k-nearest neighbor, boosting, decision trees, random forests, neural networks, pattern recognition, Bayesian networks and Hidden Markov models. Other classifiers, including improvements or combinations of any of these, commonly used for supervised learning, can also be suitable for use with the methods described herein.

Classification using supervised methods can generally be performed by the following methodology:
1. Gather a training set. These can include, for example, clinical features described herein from a sample from a patient responding or not responding to anti-TNF therapy. The training samples are used to “train” the classifier.
2. Determine the input “feature” representation of the learned function. The accuracy of the learned function depends on how the input object is represented. Typically, the input object is transformed into a feature vector, which contains a number of features that are descriptive of the object. The features may include clinical features of a patient or subject.
3. Determine the structure of the learned function and corresponding learning algorithm. A learning algorithm is chosen, e.g., artificial neural networks, decision trees, Bayes classifiers or support vector machines. The learning algorithm is used to build the classifier.
4. Build the classifier (e.g., classification model). The learning algorithm is run on the gathered training set. Parameters of the learning algorithm may be adjusted by optimizing performance on a subset (called a validation set) of the training set, or via cross-validation. After parameter adjustment and learning, the performance of the algorithm may be measured on a test set of naive samples that is separate from the training set. The built model can involve feature coefficients or importance measures assigned to individual features.
In some cases, the individual features are clinical features. In some cases, the clinical feature is a normalized value, an average value, a median value, a mean value, an adjusted average, or other adjusted level or value.

Once the classifier (e.g., classification model) is determined as described above (“trained”), it can be used to classify a sample, e.g., clinical features that are analyzed or processed according to methods described herein.

The trained model and the associated machine learning and application of the model will utilize processors, modules, memories, databases, networks, and potentially user interfaces to show the results and allow changes to be made.

In communications and computing, a computer readable medium is a medium capable of storing data in a format readable by a mechanical device. The term “non-transitory” is used herein to refer to computer readable media (“CRM”) that store data for short periods or in the presence of power such as a memory device.

One or more embodiments described herein can be implemented using programmatic modules, engines, or components. A programmatic module, engine, or component can include a program, a sub-routine, a portion of a program, or a software component or a hardware component capable of performing one or more stated tasks or functions. A module or component can exist on a hardware component independently of other modules or components. Alternatively, a module or component can be a shared element or process of other modules, programs, or machines.

The system will preferably include an intelligent control (i.e., a controller) and components for establishing communications. Examples of such a controller may be processing units alone or other subcomponents of computing devices. The controller can also include other components and can be implemented partially or entirely on a semiconductor (e.g., a field-programmable gate array (“FPGA”)) chip, such as a chip developed through a register transfer level (“RTL”) design process.

A processing unit, also called a processor, is an electronic circuit which performs operations on some external data source, usually memory or some other data stream. Non-limiting examples of processors include a microprocessor, a microcontroller, an arithmetic logic unit (“ALU”), and most notably, a central processing unit (“CPU”). A CPU, also called a central processor or main processor, is the electronic circuitry within a computer that carries out the instructions of a computer program by performing the basic arithmetic, logic, controlling, and input/output (“I/O”) operations specified by the instructions. Processing units are common in tablets, telephones, handheld devices, laptops, user displays, smart devices (TV, speaker, watch, etc.), and other computing devices.

The memory includes, in some embodiments, a program storage area and/or data storage area. The memory can comprise read-only memory (“ROM”, an example of non-volatile memory, meaning it does not lose data when it is not connected to a power source) or random-access memory (“RAM”, an example of volatile memory, meaning it will lose its data when not connected to a power source). Examples of volatile memory include static RAM (“SRAM”), dynamic RAM (“DRAM”), synchronous DRAM (“SDRAM”), etc. Examples of non-volatile memory include electrically erasable programmable read only memory (“EEPROM”), flash memory, hard disks, SD cards, etc. In some embodiments, the processing unit, such as a processor, a microprocessor, or a microcontroller, is connected to the memory and executes software instructions that are capable of being stored in a RAM of the memory (e.g., during execution), a ROM of the memory (e.g., on a generally permanent basis), or another non-transitory computer readable medium such as another memory or a disc.

Generally, the non-transitory computer readable medium operates under control of an operating system stored in the memory. The non-transitory computer readable medium implements a compiler which allows a software application written in a programming language such as COBOL, C++, FORTRAN, or any other known programming language to be translated into code readable by the central processing unit. After completion, the central processing unit accesses and manipulates data stored in the memory of the non-transitory computer readable medium using the relationships and logic dictated by the software application and generated using the compiler.

In one embodiment, the software application and the compiler are tangibly embodied in the computer-readable medium. When the instructions are read and executed by the non-transitory computer readable medium, the non-transitory computer readable medium performs the steps necessary to implement and/or use the present invention. A software application, operating instructions, and/or firmware (semi-permanent software programmed into read-only memory) may also be tangibly embodied in the memory and/or data communication devices, thereby making the software application a product or article of manufacture according to the present invention.

The database is a structured set of data typically held in a computer. The database, as well as data and information contained therein, need not reside in a single physical or electronic location. For example, the database may reside, at least in part, on a local storage device, in an external hard drive, on a database server connected to a network, on a cloud-based storage system, in a distributed ledger (such as those commonly used with blockchain technology), or the like.

It is envisioned that the machine learned model and any of the training of the same could include cloud computing. Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service.

A cloud computing environment is service oriented with a focus on statelessness, low coupling, modularity, and semantic interoperability. At the heart of cloud computing is an infrastructure comprising a network of interconnected nodes.

As noted, the training model could be implemented on a user interface. The interface could also be a point on introduction of data, such as training data or test data to compare to the trained model for analysis. The results of the comparison could then be shown on a user interface.

A user interface is how the user interacts with a machine. The user interface can be a digital interface, a command-line interface, a graphical user interface (“GUI”), oral interface, virtual reality interface, or any other way a user can interact with a machine (user-machine interface). For example, the user interface (“UI”) can include a combination of digital and analog input and/or output devices or any other type of UI input/output device required to achieve a desired level of control and monitoring for a device. Examples of input and/or output devices include computer mice, keyboards, touchscreens, knobs, dials, switches, buttons, speakers, microphones, LIDAR, RADAR, etc. Input(s) received from the UI can then be sent to a microcontroller to control operational aspects of a device.

The user interface module can include a display, which can act as an input and/or output device. More particularly, the display can be a liquid crystal display (“LCD”), a light-emitting diode (“LED”) display, an organic LED (“OLED”) display, an electroluminescent display (“ELD”), a surface-conduction electron emitter display (“SED”), a field-emission display (“FED”), a thin-film transistor (“TFT”) LCD, a bistable cholesteric reflective display (i.e., e-paper), etc. The user interface also can be configured with a microcontroller to display conditions or data associated with the main device in real-time or substantially real-time.

Any components of the system could be connected via network or other communication protocol to transfer information, communicate with other systems, or provide other connectivity. In some embodiments, the network is, by way of example only, a wide area network (“WAN”) such as a TCP/IP based network or a cellular network, a local area network (“LAN”), a neighborhood area network (“NAN”), a home area network (“HAN”), or a personal area network (“PAN”) employing any of a variety of communication protocols, such as Wi-Fi, Bluetooth, ZigBee, near field communication (“NFC”), etc., although other types of networks are possible and are contemplated herein. The network typically allows communication between the communications module and the central location during moments of low-quality connections. Communications through the network can be protected using one or more encryption techniques, such as those techniques provided by the Advanced Encryption Standard (AES), which superseded the Data Encryption Standard (DES), the IEEE 802.1 standard for port-based network security, pre-shared key, Extensible Authentication Protocol (“EAP”), Wired Equivalent Privacy (“WEP”), Temporal Key Integrity Protocol (“TKIP”), Wi-Fi Protected Access (“WPA”), and the like.

Utilizing computational algorithms, the workflow can analyze the interaction between viral proteins and host receptor proteins. This analysis reveals key entry proteins critical for viral infection. Based on the interaction analysis, epitope proteins of the virus can be identified. These proteins serve as targets for immunogen synthesis. The identified epitope proteins are used to generate immunogens, short protein fragments containing the most potent parts of viral proteins. These immunogens form the basis for vaccine design. Using in silico testing, the synthesized immunogens can be ranked as per importance and potency to be tested in vitro and in vivo to assess their efficacy in eliciting immune responses and conferring immunity against viral infection. Beneficially, in silico testing to identify the most important candidates significantly increases the efficiency of vaccine development.

Methods of the present disclosure can comprise identifying or more viral proteins and identifying one or more host receptor proteins. Said identifying can be performed through a literature search or through databases and programs such as NCBI RefSeq, RAST, and the like. In some embodiments, the viral and host genome is annotated to identify one or more proteins. Beneficially, literature reviews can identify interactions and key epitopes that may be missed by existing tools due to dataset biases, particularly for animal viruses.

The methods further comprise performing subcellular localization of the one or more viral proteins and the one or more host receptor proteins to identify one or more transmembrane viral proteins and one or more transmembrane host receptor proteins. The subcellular localization can be performed using consensus bioinformatics in which the results of multiple tools and/or programs are compared. In some embodiments, the subcellular localization step is performed using multiple tools, including, for example, BUSCA, Deep TMHMM, and/or DeepLoc. It should be understood that performance of this step is not limited to the tools recited herein, but rather any tool or method known in the art for performing subcellular localization may be used. In some embodiments, if multiple tools localize the protein as a transmembrane protein, the localization is confirmed.

The three dimensional structure of the one or more transmembrane viral proteins and the one or more transmembrane host receptor proteins can be predicted using artificial intelligence predictors such as, for example, ESMfold and/or Alphafold2. It should be understood that performance of this step is not limited to the tools recited herein, but rather any tool or method known in the art for predicting the three dimensional structure of a protein may be used.

Using the predicted three-dimensional structures, protein-protein docking poses of the of the one or more transmembrane viral proteins to the one or more transmembrane host receptor proteins can also be predicted. In some embodiments, tools such as HADDOCK 2.4, ClusPro 2.0, and/or GRAMM: Docking are used. However, it should be understood that performance of this step is not limited to the tools recited herein, but rather any tool or method known in the art for predicting docking positions of proteins may be used. In some embodiments, two, three, or more tools are used and epitopes are retained if they are captured by at least two tools. Beneficially, this can enhance result reliability. Binding and docking studies can identify viral non-structural proteins (NSPs) that interact with the host. This can provide a more accurate and comprehensive understanding of interactions, enhancing result reliability.

The predicted protein-protein docking poses can be analyzed to identify one or more viral epitopes and/or one or more host receptor paratopes. In some embodiments, results from multiple docking tools are combined and evaluated to find the common epitopes and/or paratopes. From this list of residues, the ones having the highest scores across measured metrics can be used to generate a database of viral epitopes. Epitopes from this database can be advanced for further testing and immunogen development.

Identified viral epitopes can be used to generate immunogens and antibodies against said epitopes. In some embodiments, immunogens can be constructed by tools such as RFdiffusion and/or ProteinMPNN. However, it should be understood that performance of this step is not limited to the tools recited herein, but rather any tool or method known in the art for immunogen construction may be used. The design of antibodies capable of binding to a specific antigen is commonplace in the art. In some embodiments, artificial intelligence tools can be used to design antibodies and evaluate affinity maturation of the antibodies. Programs such as, for example, IgFold, use artificial intelligence to predict antibody structure and are useful in the presently disclosed methods. Programs such as, for example, OptMAVEn-2.0, are capable of de novo design of monoclonal antibody variable regions targeting a specific antigen epitope and are useful in the presently disclosed methods. However, it should be understood that performance of this step is not limited to the tools recited herein, but rather any tool or method known in the art for antibody design and evaluation may be used.

### Immunogenic Compositions

The present disclosure provides immunogenic compositions, suitable for use as vaccines and/or treatments, including immunotherapy, against infection by porcine reproductive and respiratory syndrome virus (PRRSV) and Infectious Bronchitis Virus (IBV) proteins. The immunogenic compositions according to the disclosure elicit a specific humoral immune response toward PRRSV or IBV.

In some embodiments, the immunogen is a multi-epitope vaccine (MEV). MEVs are peptide-based vaccines comprising T cell and B cell epitopes (i.e., multiple fragments of antigenic proteins strung together by linker amino acids), designed to elicit potent cellular and humoral immune responses. MEVs represent a promising approach for combating viral infections by eliciting a comprehensive immune response. These vaccines leverage T cell receptor (TCR) recognized Major Histocompatibility Complex (MHC)-restricted epitopes from several target antigens. MEVs also demonstrate enhanced immunogenicity because they pack in more than one viral antigenic epitope. Additionally, they provide long-lasting immune protection minimizing the side effects commonly associated with traditional vaccines. In some embodiments, the MEV comprises at least one T cell epitope and at least on B cell epitope. In some embodiments, the MEV comprises adjuvant and linker sequences. In some embodiments, a database of MEVs can be generated. Such databases can be generated using machine-learning-based protein diffusion models, such as RFDiffusion. However, it should be understood that performance of this step is not limited to the tools recited herein, but rather any tool or method known in the art for protein database generation may be used.

The immunogenic and vaccine compositions of this disclosure are not, however, restricted to any particular type or method of preparation. These include, but are not limited to, infectious DNA vaccines (i.e., using plasmids, vectors or other conventional carriers to directly inject DNA into a subject), live vaccines, modified live vaccines, inactivated vaccines, subunit vaccines, attenuated vaccines, genetically engineered vaccines, etc. Furthermore, the methods disclosed herein may be used to predict viral escape mutants, allowing for predictive vaccine development. Thus, the immunogenic compositions of the present disclosure also encompass vaccines protective against such escape mutants.

In certain embodiments, the PRRSV immunogenic composition comprises one or more antigenic fragments of E envelope protein, GP2, GP3, GP4, GP5, membrane protein M, nsp3, nsp5, and/or ORF5a of PRRSV; and a pharmaceutically acceptable carrier. In some embodiments, the immunogenic composition comprises an antigenic fragment of ORF5a. In some embodiments, the antigenic fragment comprises a polypeptide of any one of SEQ ID NOs: 1-61 or a functional variant thereof. In some embodiments, the antigenic fragment comprises a polypeptide having about 70%, about 75%, about 80%, about 85%, about 90%, about 95%, or about 100% sequence identity to any one of SEQ ID NOs: 1-61. In some embodiments, the immunogenic composition comprises more than one antigenic fragment and/or polypeptide.

In some embodiments, the IBV immunogen is a MEV. The MEV can comprise at least two antigenic fragments of an IBV protein, wherein the antigenic fragments comprise at least one B-cell epitope and at least one T-cell epitope. In embodiments, the MEV comprises an amino acid sequence having at least 70%, about 75%, at least 80%, at least 85%, at least 90%, at least 95%, or at least 100% sequence identity to SEQ ID NO: 62 or 63.

### A. Immunologically Functional Equivalents

As modifications and changes may be made in the structure of an immunogenic composition of the present disclosure, and still obtain molecules having like or otherwise desirable characteristics, such functional equivalents are also encompassed within the present disclosure.

For example, certain amino acids may be substituted for other amino acids in a peptide, polypeptide or protein structure without appreciable loss of interactive binding capacity with structures such as, for example, antigen-binding regions of antibodies, binding sites on substrate molecules or receptors, DNA binding sites, or such like. Since it is the interactive capacity and nature of a peptide, polypeptide or protein that defines its biological (e.g., immunological) functional activity, certain amino acid sequence substitutions can be made in an amino acid sequence (or, of course, its underlying DNA coding sequence) and nevertheless obtain a peptide or polypeptide with like (agonistic) properties. It is thus contemplated by the inventors that various changes may be made in the sequence of an antigenic composition such as, for example a viral peptide or polypeptide without appreciable loss of biological utility or activity. In particular cases, there are one or more other amino acids that are modified compared to the corresponding wild-type sequence.

In terms of immunologically functional equivalent, it is well understood by the skilled artisan that, inherent in the definition is the concept that there is a limit to the number of changes that may be made within a defined portion of the molecule and still result in a molecule with an acceptable level of equivalent immunological activity. An immunologically functional equivalent peptide or polypeptide are thus defined herein as those peptide(s) or polypeptide(s) in which certain, not most or all, of the amino acid(s) may be substituted.

In particular, where a shorter length peptide is concerned, it is contemplated that fewer amino acid substitutions should be made within the given peptide. A longer polypeptide may have an intermediate number of changes. The full length protein will have the most tolerance for a larger number of changes. Of course, a plurality of distinct polypeptides/peptides with different substitutions may easily be made and used in accordance with the disclosure.

It also is well understood that where certain residues are shown to be particularly important to the immunological or structural properties of a protein or peptide, e.g., residues in binding regions or active sites, such residues may not generally be exchanged. This is an important consideration in the present disclosure, where changes in the antigenic site should be carefully considered and subsequently tested to ensure maintenance of immunological function (e.g., antigenicity), where maintenance of immunological function is desired. In this manner, functional equivalents are defined herein as those peptides or polypeptides which maintain a substantial amount of their native immunological activity.

Amino acid substitutions are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as immunologically functional equivalents.

To effect more quantitative changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein, polypeptide or peptide is generally understood in the art (Kyte & Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within .+−. 2 is preferred, those which are within .+−. 1 are particularly preferred, and those within .+−. 0.5 are even more particularly preferred.

It also is understood in the art that the substitution of like amino acids can be made effectively on the basis of hydrophilicity, particularly where the immunological functional equivalent polypeptide or peptide thereby created is intended for use in immunological embodiments, as in certain embodiments of the present disclosure. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a immunological property of the protein.

As detailed in U.S. Pat. No. 4,554,101, the following hydrophilicity values have been assigned to amino acid residues: arginine (+3.0); lysine (+3.0); aspartate (+3.0.+−. 1); glutamate (+3.0.+−. 1); serine (+0.3); asparagine (+0.2); glutamine (+0.2); glycine (0); threonine (−0.4); proline (−0.5.+−. 1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within .+−. 2 is preferred, those which are within .+−. 1 are particularly preferred, and those within .+−. 0.5 are even more particularly preferred.

While discussion has focused on functionally equivalent polypeptides arising from amino acid changes, it will be appreciated that these changes may be effected by alteration of the encoding DNA; taking into consideration also that the genetic code is degenerate and that two or more codons may code for the same amino acid. Nucleic acids encoding these antigenic compositions also can be constructed and inserted into one or more expression vectors by standard methods (Sambrook et al., 1987), for example, using PCR cloning methodology.

In addition to the peptidyl compounds described herein, the inventors also contemplate that other sterically similar compounds may be formulated to mimic the key portions of the peptide or polypeptide structure or to interact specifically with, for example, an antibody. Such compounds, which may be termed peptidomimetics, may be used in the same manner as a peptide or polypeptide of the disclosure and hence are also immunologically functional equivalents.

Certain mimetics that mimic elements of protein secondary structure are described in Johnson et al. (1993). The underlying rationale behind the use of peptide mimetics is that the peptide backbone of proteins exists chiefly to orientate amino acid side chains in such a way as to facilitate molecular interactions, such as those of antibody and antigen. A peptide mimetic is thus designed to permit molecular interactions similar to the natural molecule.

### B. Vaccine or Immunogenic Composition Component Purification

A vaccine component (e.g., an antigenic peptide or polypeptide) may be isolated and/or purified from the chemical synthesis reagents, cell or cellular components. In a method of producing the vaccine or immunogenic composition component, purification is accomplished by any appropriate technique that is described herein or well-known to those of skill in the art (e.g., Sambrook et al., 1987). There is no general requirement that an antigenic composition of the present disclosure or other vaccine component always be provided in their most purified state. Indeed, it is contemplated that less substantially purified vaccine or immunogenic composition component, which is nonetheless enriched in the desired compound, relative to the natural state, will have utility in certain embodiments, such as, for example, total recovery of protein product, or in maintaining the activity of an expressed protein. However, it is contemplated that inactive products also have utility in certain embodiments, such as, e.g., in determining antigenicity via antibody generation.

Various techniques suitable for use in chemical, biomolecule or biological purification, well known to those of skill in the art, may be applicable to preparation of a vaccine component of the present disclosure. These include, for example, precipitation with ammonium sulfate, PEG, antibodies and the like or by heat denaturation, followed by centrifugation; fractionation, chromatographic procedures, including but not limited to, partition chromatograph (e.g., paper chromatograph, thin-layer chromatograph (TLC), gas-liquid chromatography and gel chromatography) gas chromatography, high performance liquid chromatography, affinity chromatography, supercritical flow chromatography ion exchange, gel filtration, reverse phase, hydroxylapatite, lectin affinity; isoelectric focusing and gel electrophoresis (see for example, Sambrook et al. 1989; and Freifelder, Physical Biochemistry, Second Edition, pages 238-246, incorporated herein by reference).

Given many DNA and proteins are known (see for example, the National Center for Biotechnology Information's GenBank™ and GenPept™ databases, or may be identified and amplified using the methods described herein, any purification method for recombinantly expressed nucleic acid or proteinaceous sequences known to those of skill in the art can now be employed. In certain aspects, a nucleic acid may be purified on polyacrylamide gels, and/or cesium chloride centrifugation gradients, or by any other means known to one of ordinary skill in the art (see for example, Sambrook et al. 1989, incorporated herein by reference). In further aspects, a purification of a proteinaceous sequence may be conducted by recombinantly expressing the sequence as a fusion protein. Such purification methods are routine in the art. This is exemplified by the generation of an specific protein-glutathione S-transferase fusion protein, expression in E. coli, and isolation to homogeneity using affinity chromatography on glutathione-agarose or the generation of a polyhistidine tag on the N- or C-terminus of the protein, and subsequent purification using Ni-affinity chromatography. In particular aspects, cells or other components of the vaccine may be purified by flow cytometry. Flow cytometry involves the separation of cells or other particles in a liquid sample, and is well known in the art (see, for example, U.S. Pat. Nos. 3,826,364, 4,284,412, 4,989,977, 4,498,766, 5,478,722, 4,857,451, 4,774,189, 4,767,206, 4,714,682, 5,160,974 and 4,661,913). Any of these techniques described herein, and combinations of these and any other techniques known to skilled artisans, may be used to purify and/or assay the purity of the various chemicals, proteinaceous compounds, nucleic acids, cellular materials and/or cells that may comprise a vaccine of the present disclosure. As is generally known in the art, it is believed that the order of conducting the various purification steps may be changed, or that certain steps may be omitted, and still result in a suitable method for the preparation of a substantially purified antigen or other vaccine component.

### C. Additional Vaccine Components

It is contemplated that an immunogenic composition of the disclosure may be combined with one or more additional components to form a more effective composition or vaccine. Non-limiting examples of additional components include, for example, one or more additional antigens, immunomodulators or adjuvants to stimulate an immune response to an antigenic composition of the present disclosure and/or the additional component(s).

### 1. Immunomodulators

For example, it is contemplated that immunomodulators can be included in the vaccine to augment a cell's or a patient's (e.g., an animal's) response. Immunomodulators can be included as purified proteins, nucleic acids encoding immunomodulators, and/or cells that express immunomodulators in the vaccine composition, for example. The following sections list non-limiting examples of immunomodulators that are of interest, and it is contemplated that various combinations of immunomodulators may be used in certain embodiments (e.g., a cytokine and a chemokine).

Interleukins, cytokines, nucleic acids encoding interleukins or cytokines, and/or cells expressing such compounds are contemplated as possible vaccine components. Interleukins and cytokines, include but are not limited to interleukin 1 (IL-1), IL-2, IL-3, IL-4, IL-5, IL-6, IL-7, IL-8, IL-9, IL-10, IL-11, IL-12, IL-13, IL-14, IL-15, IL-18, .beta.-interferon, .alpha.-interferon, .gamma.-interferon, angiostatin, thrombospondin, endostatin, GM-CSF, G-CSF, M-CSF, METH-1, METH-2, tumor necrosis factor, TGF .beta., LT and combinations thereof.

Chemokines, nucleic acids that encode for chemokines, and/or cells that express such also may be used as vaccine components. Chemokines generally act as chemoattractants to recruit immune effector cells to the site of chemokine expression. It may be advantageous to express a particular chemokine coding sequence in combination with, for example, a cytokine coding sequence, to enhance the recruitment of other immune system components to the site of treatment. Such chemokines include, for example, RANTES, MCAF, MIP1-alpha, MIP1-Beta, IP-10 and combinations thereof. The skilled artisan will recognize that certain cytokines are also known to have chemoattractant effects and could also be classified under the term chemokines.

In certain embodiments, an antigenic composition may be chemically coupled to a carrier or recombinantly expressed with a immunogenic carrier peptide or polypetide (e.g., a antigen-carrier fusion peptide or polypeptide) to enhance an immune reaction. Exemplary and preferred immunogenic carrier amino acid sequences include hepatitis B surface antigen, keyhole limpet hemocyanin (KLH) and bovine serum albumin (BSA). Other albumins such as ovalbumin, mouse serum albumin or rabbit serum albumin also can be used as immunogenic carrier proteins. Means for conjugating a polypeptide or peptide to a immunogenic carrier protein are well known in the art and include, for example, glutaraldehyde, m-maleimidobenzoyl-N-hydroxysuccinimide ester, carbodiimide and bis-biazotized benzidine.

It may be desirable to coadminister biologic response modifiers (BRM), which have been shown to upregulate T cell immunity or downregulate suppressor cell activity. Such BRMs include, but are not limited to, cimetidine (CIM; 1200 mg/d) (Smith/Kline, PA); low-dose cyclophosphamide (CYP; 300 mg/m.sup.2) (Johnson/Mead, NJ), or a gene encoding a protein involved in one or more immune helper functions, such as B-7.

### 2. Adjuvants

Immunization protocols have used adjuvants to stimulate responses for many years, and as such adjuvants are well known to one of ordinary skill in the art. Some adjuvants affect the way in which antigens are presented. For example, the immune response is increased when protein antigens are precipitated by alum. Emulsification of antigens also prolongs the duration of antigen presentation. In one aspect, an adjuvant effect is achieved by use of an agent, such as alum, used in about 0.05 to about 0.1% solution in phosphate buffered saline. Alternatively, the antigen is made as an admixture with synthetic polymers of sugars (Carbopol™) used as an about 0.25% solution. Adjuvant effect may also be made my aggregation of the antigen in the vaccine by heat treatment with temperatures ranging between about 70° to about 101° C. for a 30-second to 2-minute period, respectively. Aggregation by reactivating with pepsin treated (Fab) antibodies to albumin, mixture with bacterial cell(s) such as C. parvum, an endotoxin or a lipopolysaccharide component of Gram-negative bacteria, emulsion in physiologically acceptable oil vehicles, such as mannide mono-oleate (Aracel A), or emulsion with a 20% solution of a perfluorocarbon (Fluosol-DA™) used as a block substitute, also may be employed.

Some adjuvants, for example, certain organic molecules obtained from bacteria, act on the host rather than on the antigen. An example is muramyl dipeptide (N-acetylmuramyl-L-alanyl-D-isoglutamine [MDP]), a bacterial peptidoglycan. The effects of MDP, as with most adjuvants, are not fully understood. MDP stimulates macrophages but also appears to stimulate B cells directly. The effects of adjuvants, therefore, are not antigen-specific. If they are administered together with a purified antigen, however, they can be used to selectively promote the response to the antigen.

Adjuvants have been used experimentally to promote a generalized increase in immunity against unknown antigens (e.g., U.S. Pat. No. 4,877,611). In certain embodiments, hemocyanins and hemoerythrins may also be used in the disclosure. The use of hemocyanin from keyhole limpet (KLH) is preferred in certain embodiments, although other molluscan and arthropod hemocyanins and hemoerythrins may be employed.

Various polysaccharide adjuvants may also be used. For example, the use of various pneumococcal polysaccharide adjuvants on the antibody responses of mice has been described (Yin et al., 1989). The doses that produce optimal responses, or that otherwise do not produce suppression, should be employed as indicated (Yin et al., 1989). Polyamine varieties of polysaccharides are particularly preferred, such as chitin and chitosan, including deacetylated chitin.

Another group of adjuvants are the muramyl dipeptide (MDP, N-acetylmuramyl-L-alanyl-D-isoglutamine) group of bacterial peptidoglycans. Derivatives of muramyl dipeptide, such as the amino acid derivative threonyl-MDP, and the fatty acid derivative MTPPE, are also contemplated.

U.S. Pat. No. 4,950,645 describes a lipophilic disaccharide-tripeptide derivative of muramyl dipeptide which is described for use in artificial liposomes formed from phosphatidyl choline and phosphatidyl glycerol. It is the to be effective in activating human monocytes and destroying tumor cells, but is non-toxic in generally high doses. The compounds of U.S. Pat. No. 4,950,645 and PCT Patent Application WO 91/16347, are contemplated for use with cellular carriers and other embodiments of the present disclosure.

Another adjuvant contemplated for use in the present disclosure is BCG. BCG (bacillus Calmette-Guerin, an attenuated strain of Mycobacterium) and BCG-cell wall skeleton (CWS) may also be used as adjuvants in the disclosure, with or without trehalose dimycolate. Trehalose dimycolate may be used itself. Trehalose dimycolate administration has been shown to correlate with augmented resistance to influenza virus infection in mice (Azuma et al., 1988). Trehalose dimycolate may be prepared as described in U.S. Pat. No. 4,579,945.

BCG is an important clinical tool because of its immunostimulatory properties. BCG acts to stimulate the reticulo-endothelial system, activates natural killer cells and increases proliferation of hematopoietic stem cells. Cell wall extracts of BCG have proven to have excellent immune adjuvant activity. Molecular genetic tools and methods for mycobacteria have provided the means to introduce foreign genes into BCG (Jacobs et al., 1987; Snapper et al., 1988; Husson et al., 1990; Martin et al., 1990).

Live BCG is an effective and safe vaccine used worldwide to prevent tuberculosis. BCG and other mycobacteria are highly effective adjuvants, and the immune response to mycobacteria has been studied extensively. With nearly 2 billion immunizations, BCG has a long record of safe use in man (Luelmo, 1982; Lotte et al., 1984). It is one of the few vaccines that can be given at birth, it engenders long-lived immune responses with only a single dose, and there is a worldwide distribution network with experience in BCG vaccination. An exemplary BCG vaccine is sold as TICE™ BCG (Organon Inc., West Orange, N.J.).

Amphipathic and surface active agents, e.g., saponin and derivatives such as QS21 (Cambridge Biotech), form yet another group of adjuvants for use with the immunogens of the present disclosure. Nonionic block copolymer surfactants (Rabinovich et al., 1994; Hunter et al., 1991) may also be employed. Oligonucleotides are another useful group of adjuvants (Yamamoto et al., 1988). Quil A and lentinen are other adjuvants that may be used in certain embodiments of the present disclosure.

One group of adjuvants preferred for use in the disclosure are the detoxified endotoxins, such as the refined detoxified endotoxin of U.S. Pat. No. 4,866,034. These refined detoxified endotoxins are effective in producing adjuvant responses in mammals. Of course, the detoxified endotoxins may be combined with other adjuvants to prepare multi-adjuvant-incorporated cells. For example, combination of detoxified endotoxins with trehalose dimycolate is particularly contemplated, as described in U.S. Pat. No. 4,435,386. Combinations of detoxified endotoxins with trehalose dimycolate and endotoxic glycolipids is also contemplated (U.S. Pat. No. 4,505,899), as is combination of detoxified endotoxins with cell wall skeleton (CWS) or CWS and trehalose dimycolate, as described in U.S. Pat. Nos. 4,436,727, 4,436,728 and 4,505,900. Combinations of just CWS and trehalose dimycolate, without detoxified endotoxins, is also envisioned to be useful, as described in U.S. Pat. No. 4,520,019.

In other embodiments, the present disclosure contemplates that a variety of adjuvants may be employed in the membranes of cells, resulting in an improved immunogenic composition. The only requirement is, generally, that the adjuvant be capable of incorporation into, physical association with, or conjugation to, the cell membrane of the cell in question. Those of skill in the art will know the different kinds of adjuvants that can be conjugated to cellular vaccines in accordance with this disclosure and these include alkyl lysophosphilipids (ALP); BCG; and biotin (including biotinylated derivatives) among others. Certain adjuvants particularly contemplated for use are the teichoic acids from Gram-cells. These include the lipoteichoic acids (LTA), ribitol teichoic acids (RTA) and glycerol teichoic acid (GTA). Active forms of their synthetic counterparts may also be employed in connection with the disclosure (Takada et al., 1995a).

Various adjuvants, even those that are not commonly used in humans, may still be employed in animals, where, for example, one desires to raise antibodies or to subsequently obtain activated T cells. The toxicity or other adverse effects that may result from either the adjuvant or the cells, e.g., as may occur using non-irradiated tumor cells, is irrelevant in such circumstances.

One group of adjuvants preferred for use in some embodiments of the present disclosure are those that can be encoded by a nucleic acid (e.g., DNA or RNA). It is contemplated that such adjuvants may be encoded in a nucleic acid (e.g., an expression vector) encoding the antigen, or in a separate vector or other construct. These nucleic acids encoding the adjuvants can be delivered directly, such as for example with lipids or liposomes.

### 3. Excipients, Salts and Auxiliary Substances

An antigenic composition of the present disclosure may be mixed with one or more additional components (e.g., excipients, salts, etc.) which are pharmaceutically acceptable and compatible with at least one active ingredient (e.g., antigen). Suitable excipients are, for example, water, saline, dextrose, glycerol, ethanol and combinations thereof.

An antigenic composition of the present disclosure may be formulated into the vaccine as a neutral or salt form. A pharmaceutically-acceptable salt, includes the acid addition salts (formed with the free amino groups of the peptide) and those which are formed with inorganic acids such as, for example, hydrochloric or phosphoric acid, or such organic acids as acetic, oxalic, tartaric, mandelic, and the like. A salt formed with a free carboxyl group also may be derived from an inorganic base such as, for example, sodium, potassium, ammonium, calcium, or ferric hydroxide, and such organic bases as isopropylamine, trimethylamine, 2-ethylamino ethanol, histidine, procaine, and combinations thereof.

In addition, if desired, an antigentic composition may comprise minor amounts of one or more auxiliary substances such as for example wetting or emulsifying agents, pH buffering agents, etc. which enhance the effectiveness of the antigenic composition or vaccine.

### D. Vaccine and Immunogenic Composition Preparations

Once produced, synthesized and/or purified, an antigen or other vaccine component may be prepared as a vaccine or immunogenic composition for administration to an individual. The preparation of a vaccine is generally well understood in the art, as exemplified by U.S. Pat. Nos. 4,608,251, 4,601,903, 4,599,231, 4,599,230, and 4,596,792, all incorporated herein by reference. Such methods may be used to prepare a vaccine comprising an antigenic composition comprising a particular viral protein as active ingredient(s), in light of the present disclosure. In particular embodiments, the compositions of the present disclosure are prepared to be pharmacologically acceptable vaccines.

Pharmaceutical vaccine or immunogenic compositions of the present disclosure comprise an effective amount of viral protein dissolved or dispersed in a pharmaceutically acceptable carrier. The phrases “pharmaceutical or pharmaceutically acceptable” refers to molecular entities and compositions that do not produce an adverse, allergic or other untoward reaction when administered to an animal, such as, for example, a human, as appropriate. The preparation of a pharmaceutical composition that contains at least one viral protein will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

Examples of pharmaceutically acceptable carriers include any and all solvents, dispersion media, diluents, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). The immunogenic composition may comprise different types of carriers depending on whether it is to be administered in solid, liquid or aerosol form, and whether it need to be sterile for such routes of administration as injection. Except insofar as any conventional carrier is incompatible with the active ingredient, its use in the therapeutic or pharmaceutical compositions is contemplated.

The immunogenic composition may be formulated in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present disclosure. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the immunogenic composition is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food or water supply of the diet. Preferred carriers for oral administration comprise inert diluents, assimilable edible carriers or combinations thereof. In other aspects of the disclosure, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein.

In particular embodiments, prolonged absorption of an injectable composition can be brought about by the use in the compositions of agents delaying absorption, such as, for example, aluminum monostearate, gelatin or combinations thereof.

### E. Vaccine or Immunogenic Composition Administration

The manner of administration of a vaccine or immunogenic composition may be varied widely. Any of the conventional methods for administration of a vaccine or immunogenic composition are applicable. For example, a vaccine may be conventionally administered intravenously, intradermally, intraarterially, intraperitoneally, intralesionally, intracranially, intraarticularly, intraprostaticaly, intrapleurally, intratracheally, intranasally, intravitreally, intravaginally, intratumorally, intramuscularly, intraperitoneally, subcutaneously, intravesicularlly, mucosally, intrapericardially, orally, rectally, nasally, topically, in eye drops, locally, using aerosol, injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990).

A vaccination or immunogenic composition delivery schedule and dosages may be varied on a patient by patient basis, taking into account, for example, factors such as the weight and age of the patient, the type of disease being treated, the severity of the disease condition, previous or concurrent therapeutic interventions, the manner of administration and the like, which can be readily determined by one of ordinary skill in the art.

A vaccine or immunogenic composition may be administered in a manner compatible with the dosage formulation, and in such amount as will be therapeutically effective and immunogenic. For example, the intramuscular route may be preferred in the case of toxins with short half lives in vivo. The quantity to be administered depends on the subject to be treated, including, e.g., the capacity of the individual's immune system to synthesize antibodies, and the degree of protection desired. The dosage of the vaccine will depend on the route of administration and will vary according to the size of the host. Precise amounts of an active ingredient required to be administered depend on the judgment of the practitioner. In certain embodiments, pharmaceutical compositions may comprise, for example, at least about 0.1% of an active compound. In other embodiments, the active compound may comprise between about 2% to about 75% of the weight of the unit, or between about 25% to about 60%, for example, and any range derivable therein. A suitable regime for initial administration and booster administrations (e.g., innoculations) are also variable, but are typified by an initial administration followed by subsequent inoculation(s) or other administration(s).

In many instances, it will be desirable to have multiple administrations of the vaccine or immunogenic composition, usually not exceeding six vaccinations, for example, more usually not exceeding four vaccinations and in some cases one or more, usually at least about three vaccinations. The vaccinations may be at from two to twelve week intervals, more usually from three to five week intervals, although longer intervals are encompassed herein. Periodic boosters may be desirable to maintain protective levels of the antibodies.

The course of the immunization may be followed by assays for antibodies for the supernatant antigens. The assays may be performed by labeling with conventional labels, such as radionuclides, enzymes, fluorescents, and the like. These techniques are well known and may be found in a wide variety of patents, such as U.S. Pat. Nos. 3,791,932; 4,174,384 and 3,949,064, as illustrative of these types of assays. Other immune assays can be performed and assays of protection from challenge with live virus can be performed, following immunization.

### Antibodies

Also contemplated by the present disclosure are anti-viral antibodies (e.g., monoclonal and polyclonal antibodies, single chain antibodies, chimeric antibodies, humanized, human, porcine, and CDR-grafted antibodies, including compounds which include CDR sequences which specifically recognize a PRRSV antigen of the disclosure. The term “specific for” indicates that the variable regions of the antibodies of the disclosure recognize and bind a PRRSV antigen exclusively (i.e., are able to distinguish a single PRRSV polypeptide from related polypeptides despite sequence identity, homology, or similarity found in the family of polypeptides), and which are permitted (optionally) to interact with other proteins (for example, S. aureus protein A or other antibodies in ELISA techniques) through interactions with sequences outside the variable region of the antibodies, and in particular, in the constant region of the Ab molecule. Traditional screening assays to determine binding specificity of an antibody of the disclosure are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies that recognize and bind fragments of PRRSV polypeptides of the disclosure are also contemplated, provided that the antibodies are first and foremost specific for, as defined above, a PRRSV polypeptide of the disclosure from which the fragment was derived.

For the purposes of clarity, “antibody” refers to an immunoglobulin molecule that can bind to a specific antigen as the result of an immune response to that antigen. Immunoglobulins are serum proteins composed of “light” and “heavy” polypeptide chains having “constant” and “variable” regions and are divided into classes (e.g., IgA, IgD, IgE, IgG, and IgM) based on the composition of the constant regions. Antibodies can exist in a variety of forms including, for example, as, Fv, Fab′, F(ab′)2, as well as in single chains, and include synthetic polypeptides that contain all or part of one or more antibody single chain polypeptide sequences.

### EXAMPLES

### Example 1. Case Study with PRRSV and Pigs

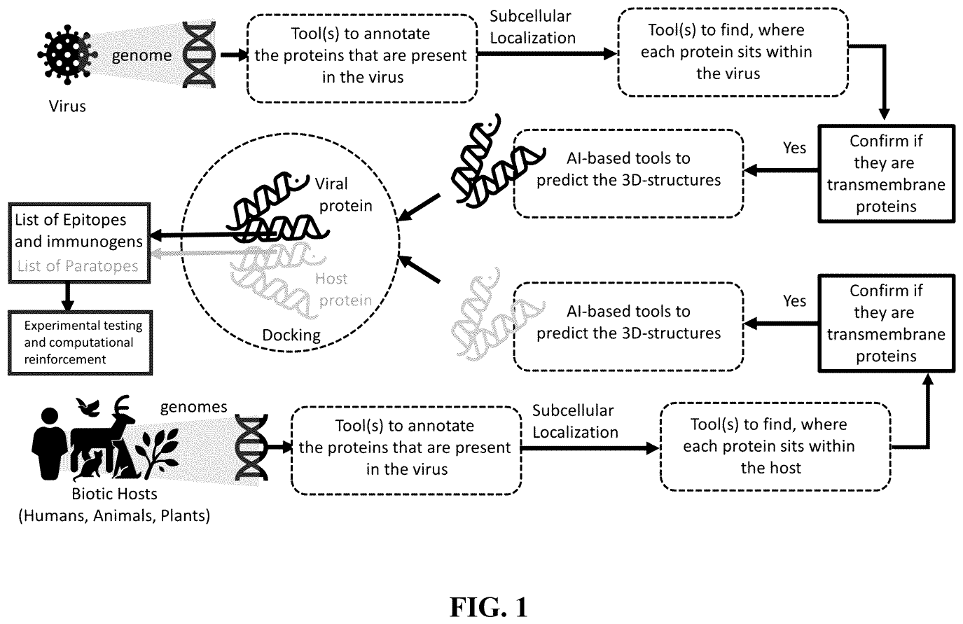

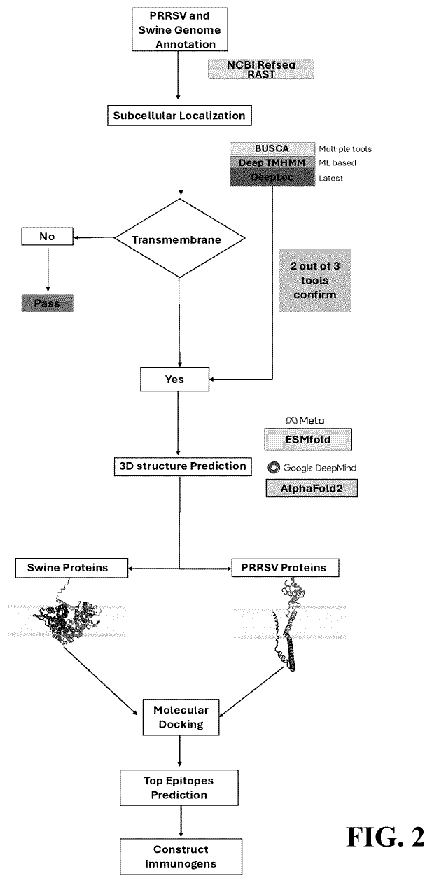

The present disclosure provides a computational assembly line or workflow for identifying epitope proteins of viruses infecting human, animal, or plant hosts via receptor proteins (FIG. 1). This workflow facilitates the identification of immunogens, short protein fragments containing potent parts of viral proteins, as a basis for antiviral vaccine development. The disclosure is demonstrated using the PRSSV-pig virus-host pair (FIG. 2), revealing key entry proteins like CD163 and identifying additional candidate proteins for vaccine synthesis, particularly relevant to the pork production industry in Iowa. Viruses often exploit host receptor proteins to gain entry and initiate infection. Identifying viral epitope proteins and designing immunogens targeting these proteins are crucial steps in developing effective antiviral vaccines. The present disclosure streamlines this process through computational analysis, reducing the time and resources required for vaccine development. The computational assembly line comprises several modules designed to analyze virus-host interactions, identify epitope proteins, and synthesize immunogens for vaccine development. The workflow includes:

Virus-Host Interaction Analysis: Utilizing computational algorithms, the workflow analyzes the interaction between viral proteins and host receptor proteins. This analysis reveals key entry proteins critical for viral infection.

Epitope Protein Identification: Based on the interaction analysis, epitope proteins of the virus are identified. These proteins serve as targets for immunogen synthesis.

Immunogen Synthesis: The identified epitope proteins are used to generate immunogens, short protein fragments containing the most potent parts of viral proteins. These immunogens form the basis for vaccine design.

In Vitro and In Vivo Experiments: The synthesized immunogens are ranked as per importance and potency to be tested in vitro and in vivo to assess their efficacy in eliciting immune responses and conferring immunity against viral infection.

The disclosure is exemplified using the PRSSV-pig virus-host pair. Computational analysis of this system reveals CD163 as a key gateway protein for PRRS virus entry into pigs. Clinical studies have already demonstrated that CD163 deletion in pigs confers immunity against PRRSV infection, validating the computational findings. Furthermore, the analysis identifies a set of candidate entry proteins that, when targeted or used as templates for antibody design, could confer similar immunity to pigs. This discovery is of significant economic importance to Iowa, the largest pork producer in the nation, as it offers a pathway to enhance disease resistance in pigs and safeguard the pork-based economy.

Therefore, the computational assembly line of the present disclosure enables efficient identification of viral epitope proteins and synthesis of immunogens for antiviral vaccine development. The demonstrated application in the PRSSV-pig system showcases the practical utility of this workflow in advancing disease intervention strategies, particularly in agriculture-dependent regions like Iowa. This patent represents a significant advancement in the field of vaccine development, providing a streamlined approach to combat viral infections in diverse host species.

The genome annotation from PRRSV identified a total of 41 proteins. Out of these, predictions from at least two tools (BUSCA, Deep TMHMM, DeepLoc) indicated that 20 of the proteins have a transmembrane region. After removing isoforms (i.e., duplicates) and unstructured proteins, we compiled a list of 9 PRRSV proteins with the 3D structure. Our literature review revealed 7 swine proteins that have been consistently found to interact with PRRSV in numerous instances (Table 1).

TABLE 1
The list and details of the PRRSV proteins and swine proteins

| PRRSV | Protein | NCBI RefSeq | Porcine | Protein | NCBI RefSeq |
|---|---|---|---|---|---|
| Proteins | Length | Accession | Proteins | Length | Accession |
| E envelope | 73 | YP_009505550.1 | CD209 | 240 | NP_001123444.1 |
| protein | | | | | |
| GP2 | 256 | NP_047408.1 | Vimentin | 466 | XP_005668163.1 |
| GP3 | 265 | A0MD32.1 | CD151 | 253 | XP_013845140.1 |
| GP4 | 183 | A0MD33.1 | Sialohedsin | 1730 | XP_020932962.1 |
| GP5 | 200 | NP_047411.1 | Heparan | 882 | XP_020940682.1 |
| | | | Sulfate | | |
| Membrane | 174 | NP_047412.1 | CD163 | 1113 | XP_020946779.1 |
| Protein M | | | | | |
| nsp3 | 231 | NP_740597.1 | MYH-9 | 1981 | XP_020947197.1 |
| nsp5 | 170 | NP_740599.1 | | | |
| ORF5a | 51 | YP_006488613.1 | | | |

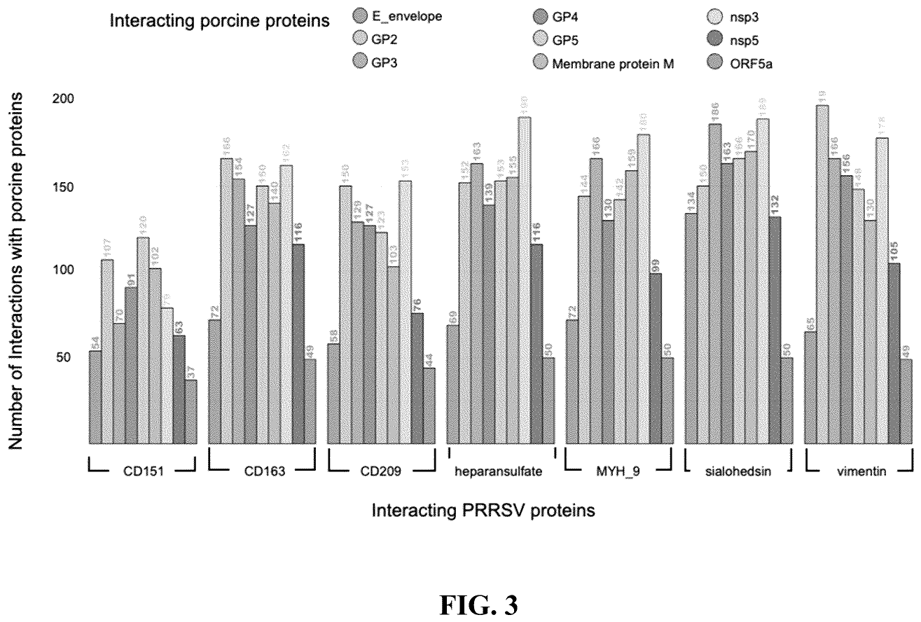

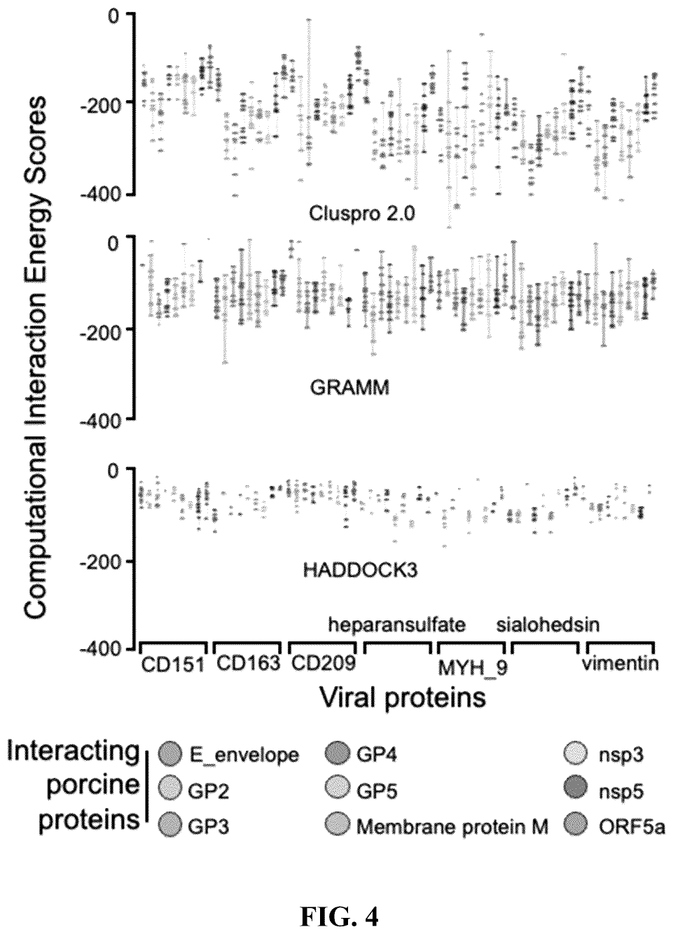

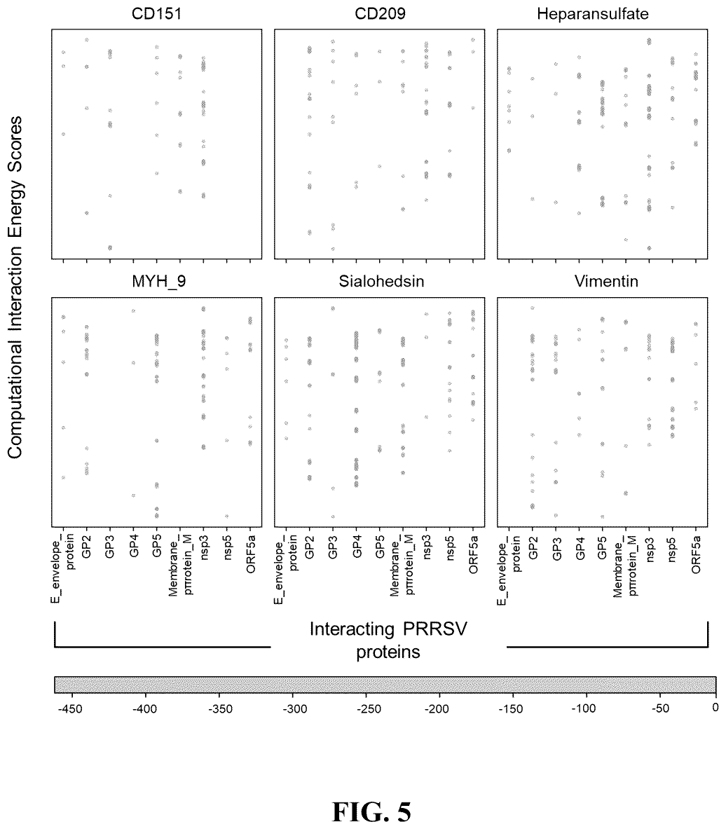

The combined results from three protein-protein docking tools HADDOCK 2.4, ClusPro 2.0, and GRAMM: Docking yielded a total of 1,696 poses (FIG. 3). It encompasses poses from HADDOCK 2.4, 630 from ClusPro 2.0, and 630 from GRAMM: Docking, for all 63 potential docked complexes formed by the 9 PRRSV proteins and 7 swine ones. We then selected of the commonly found interacting PRRSV residues by all three of the tools, which total led to 7686 unique residues. Among them, we selected the top residues for 6 of the swine proteins with the counterpart PRRSV ones. This top residue selection was done by comparing Haddock_energy_score, which led to altogether 75 residues (FIGS. 4-5 and Table 2).

TABLE 2
Predicted Viral Epitopes

| PRRSV Protein/Docking | Identified Viral |
|---|---|
| Position/Porcine Protein | Epitope Positions |
| GP2_1.pos1CD209.pdb | MET19, PHE16 |
| GP2_1.pos4CD209.pdb | SER215, ILE211, PRO5 |
| GP2_2.pos10vimentin.pdb | LEU39, VAL241 |
| GP2_2.pos7vimentin.pdb | VAL182 |
| GP2_2.pos9vimentin.pdb | CYS38, PRO27, THR226 |
| GP2_3.pos1CD151.pdb | ARG22, MET19 |
| GP3_1.pos1CD209.pdb | PRO223 |
| GP3_1.pos2CD209.pdb | GLY175 |
| GP3_1.pos3CD209.pdb | VAL20 |
| GP3_2.pos8vimentin.pdb | SER28 |
| GP3_3.pos10CD151.pdb | ILE16, PHE8, HIS9, ALA6 |
| GP3_3.pos2CD151.pdb | CYS5 |
| GP3_4.pos2sialohedsin.pdb | CYS5 |
| GP3_4.pos4sialohedsin.pdb | PHE8 |
| GP4_4.pos1sialohedsin.pdb | ASN97, ASN134, THR136, |
| | ASP137, ILE15, HIS155, |
| | HIS30, GLY11, LYS69, |
| | SER70, SER71, GLN72 |
| GP4_5.pos6heparansulfate.pdb | VAL79 |
| GP4_7.pos8MYH-9.pdb | GLY11 |
| GP5_2.pos2vimentin.pdb | ASP54 |
| GP5_7.pos10MYH-9.pdb | SER184, TYR106, LEU50 |
| GP5_7.pos3MYH-9.pdb | VAL102, VAL114, VAL107, |
| | ALA29 |
| GP5_7.pos4MYH-9.pdb | ALA57 |
| membrane_protein_M_1.pos6CD209.pdb | GLY150, ASN69 |
| membrane_protein_M_2.pos2vimentin.pdb | GLY58, ALA54 |
| membrane_protein_M_5.pos10heparansulfate.pdb | THR68 |
| nsp3_1.pos1CD209.pdb | LEU139 |
| nsp3_3.pos1CD151.pdb | ALA12, CYS13, ALA16, |
| nsp3_5.pos2heparansulfate.pdb | SER100, PRO223 |
| nsp3_5.pos3heparansulfate.pdb | SER224, PRO138, LEU142 |
| nsp3_5.pos5heparansulfate.pdb | ALA12, ALA8, LEU70, LEU5, |
| | HIS4, HIS97, LEU66, HIS64, |
| | GLY65, LEU66, LEU139, |
| | GLN63, THR140 |
| nsp5_5.pos8heparansulfate.pdb | LEU143 |
| nsp5_7.pos10MYH-9.pdb | GLY20 |

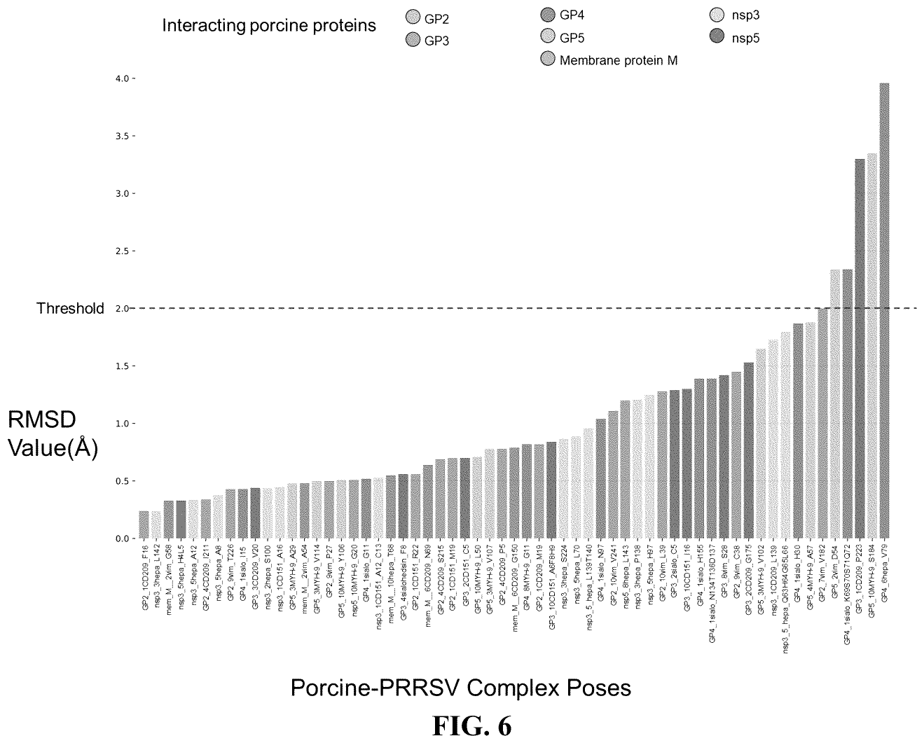

Following the identification of the primary epitopes, we formulated immunogens by selecting residues −5 to +5 amino acids around the chosen epitopes and assessing their stability in isolation from the original viral protein (Table 3). A lower RMSD (closer to zero) is associated with increased stability. Conversely, a higher RMSD indicates that the immunogen is less stable. In instances where the top epitopes overlapped within 2 residues of each other, we designed elongated immunogens that spanned across these proximate residues in the same manner. After doing relaxing simulation, we constructed top 61 immunogens (FIG. 6, SEQ ID NOs: 1-61). The immunogens, particularly those with the lowest RMSD values (i.e., the most stable), are ideal candidates for animal vaccination trials.

TABLE 3
Structural Stability of Predicted Docking

| | RMSD |
|---|---|
| Reference Structure | (√Ö) |
| GP2_2.pos9vimentin.pdb_THR226_residues_chain_B.pdb | 0.43 |
| GP4_4.pos1sialohedsin.pdb_ASN97_residues_chain_B.pdb | 1.04 |
| nsp3_5.pos5heparansulfate.pdb_ALA8_residues_chain_B.pdb | 0.38 |
| GP5_7.pos3MYH-9.pdb_VAL107_residues_chain_B.pdb | 0.78 |
| membrane_protein_M_2.pos2vimentin.pdb_ALA54_residues_chain_B.pdb | 0.48 |
| membrane_protein_M_1.pos6CD209.pdb_GLY150_residues_chain_B.pdb | 0.79 |
| GP3_3.pos10CD151.pdb_ILE16_residues_chain_B.pdb | 1.3 |
| nsp3_5.pos3heparansulfate.pdb_PRO138_residues_chain_B.pdb | 1.21 |
| GP4_4.pos1sialohedsin_N134T136D137.pdb | 1.39 |
| GP3_3.pos10CD151_A6F8H9.pdb | 0.84 |
| nsp3_5.pos3heparansulfate.pdb_SER224_residues_chain_B.pdb | 0.87 |
| nsp3_5.pos3heparansulfate.pdb_LEU142_residues_chain_B.pdb | 0.24 |
| GP4_4.pos1sialohedsin.pdb_ILE15_residues_chain_B.pdb | 0.43 |
| GP5_2.pos2vimentin.pdb_ASP54_residues_chain_B.pdb | 2.34 |
| GP2_2.pos10vimentin.pdb_VAL241_residues_chain_B.pdb | 1.11 |
| GP5_7.pos10MYH-9.pdb_LEU50_residues_chain_B.pdb | 0.71 |
| GP5_7.pos10MYH-9.pdb_TYR106_residues_chain_B.pdb | 0.51 |
| GP2_1.pos4CD209.pdb_SER215_residues_chain_B.pdb | 0.69 |
| membrane_protein_M_1.pos6CD209.pdb_ASN69_residues_chain_B.pdb | 0.64 |
| nsp3_3.pos1CD151_A12_C13.pdb | 0.53 |
| nsp3_3.pos1CD151.pdb_ALA16_residues_chain_B.pdb | 0.45 |
| GP5_7.pos10MYH-9.pdb_SER184_residues_chain_B.pdb | 3.35 |
| GP3_1.pos3CD209.pdb_VAL20_residues_chain_B.pdb | 0.44 |
| GP3_1.pos1CD209.pdb_PRO223_residues_chain_B.pdb | 3.3 |
| GP3_1.pos2CD209.pdb_GLY175_residues_chain_B.pdb | 1.53 |
| GP2_2.pos7vimentin.pdb_VAL182_residues_chain_B.pdb | 2 |
| GP2_3.pos1CD151.pdb_ARG22_residues_chain_B.pdb | 0.56 |
| GP3_3.pos2CD151.pdb_CYS5_residues_chain_B.pdb | 0.7 |
| GP5_7.pos3MYH-9.pdb_VAL114_residues_chain_B.pdb | 0.5 |
| nsp3_5.pos5heparansulfate.pdb_LEU70_residues_chain_B.pdb | 0.89 |
| GP2_1.pos1CD209.pdb_MET19_residues_chain_B.pdb | 0.82 |
| GP4_4.pos1sialohedsin.pdb_GLY11_residues_chain_B.pdb | 0.52 |
| GP2_2.pos10vimentin.pdb_LEU39_residues_chain_B.pdb | 1.28 |
| GP2_2.pos9vimentin.pdb_PRO27_residues_chain_B.pdb | 0.5 |
| GP2_1.pos1CD209.pdb_PHE16_residues_chain_B.pdb | 0.24 |
| nsp3_5.pos5_heparansulfate_L139T140.pdb | 0.96 |
| membrane_protein_M_2.pos2vimentin.pdb_GLY58_residues_chain_B.pdb | 0.33 |
| GP4_4.pos1sialohedsin.pdb_HIS30_residues_chain_B.pdb | 1.87 |
| GP4_7.pos8MYH-9.pdb_GLY11_residues_chain_B.pdb | 0.82 |
| GP3_4.pos4sialohedsin.pdb_PHE8_residues_chain_B.pdb | 0.56 |
| nsp3_5.pos5heparansulfate.pdb_ALA12_residues_chain_B.pdb | 0.34 |
| membrane_protein_M_5.pos10heparansulfate.pdb_THR68_residues_chain_B.pdb | 0.55 |
| GP2_1.pos4CD209.pdb_ILE211_residues_chain_B.pdb | 0.34 |
| nsp3_5.pos2heparansulfate.pdb_SER100_residues_chain_B.pdb | 0.44 |
| nsp3_5.pos5_heparansulfate_Q63H64G65L66.pdb | 1.8 |
| GP2_2.pos9vimentin.pdb_CYS38_residues_chain_B.pdb | 1.45 |
| nsp3_1.pos1CD209.pdb_LEU139_residues_chain_B.pdb | 1.73 |
| GP3_2.pos8vimentin.pdb_SER28_residues_chain_B.pdb | 1.42 |
| GP2_3.pos1CD151.pdb_MET19_residues_chain_B.pdb | 0.7 |
| GP5_7.pos4MYH-9.pdb_ALA57_residues_chain_B.pdb | 1.88 |
| GP4_4.pos1sialohedsin_K69S70S71Q72.pdb | 2.34 |
| GP4_4.pos1sialohedsin.pdb_HIS155_residues_chain_B.pdb | 1.39 |
| nsp5_7.pos10MYH-9.pdb_GLY20_residues_chain_B.pdb | 0.51 |
| nsp_5.pos5heparansulfate_H4L5.pdb | 0.33 |
| nsp5_5.pos8heparansulfate.pdb_LEU143_residues_chain_B.pdb | 1.2 |
| GP5_7.pos3MYH-9.pdb_ALA29_residues_chain_B.pdb | 0.48 |
| GP2_1.pos4CD209.pdb_PRO5_residues_chain_B.pdb | 0.78 |
| nsp3_5.pos5heparansulfate.pdb_HIS97_residues_chain_B.pdb | 1.25 |
| GP3_4.pos2sialohedsin.pdb_CYS5_residues_chain_B.pdb | 1.29 |
| GP4_5.pos6heparansulfate.pdb_VAL79_residues_chain_B.pdb | 3.96 |

Porcine reproductive and respiratory syndrome (PRRS) has been known to be one of the most economically impactful swine infectious disease. Annually, it is responsible for over billion-dollar losses to the worldwide pork industry. PRRSV infection causes severe reproductive failure in sows and respiratory diseases in piglets, which is further complicated by several secondary infections. This results in higher morality and clinical manifestations. PRRSV vaccines have been accessible in North America for nearly three decades. However, obstacles persist because the vaccines cannot generate comprehensive protection. This is primarily due to the variability of PRRSV and the intricate nature of the interplay between PRRSV and the immune responses of the host. Overcoming these obstacles will require more exploration of how virus and hosts interact. Overall, current strategies for controlling PRRSV have been mostly insufficient.

PRRSV can escape from the host immune response in various processes. Many novel strategies are being proposed to create more effective vaccines against this evolving virus. The diversity of PRRSV is significantly influenced by high-frequency mutation and recombination between different lineages/sub-lineages. This phenomenon holds considerable importance in the acceleration of the evolution of PRRSV. Antibody escape mutations influences the rates of viral reinfection and the duration of vaccine effectiveness. Therefore, the key to developing optimal vaccines and therapeutics depends on anticipating viral variants that can elude immune detection with enough lead time.

Ideally, experimental methods such as pseudovirus assay can help in anticipating viral immune assay, but that limits the impact for predicting immune escape early. Therefore, it is of interest to develop computational methods that can predict viral escape. This information can be used to predict better vaccines for the virus. Moreover, an ideal model would be able to consider as-yet-unseen variation of antigen and antibody interaction throughout the full antigenic protein and would guide the formulations of specific experiments. In this context, artificial intelligence (AI), and machine learning (ML), hold significant promise for expediting and enhancing the optimization of therapeutics. Artificial intelligence (AI) has the capacity to design antibodies without the necessity for screening and developing lead molecules, leading to a reduction in the time and resources required for the development of therapeutic antibodies. Overall, it has the potential to significantly enhance the speed, quality, and controllability of antibody design. Antibodies designed by AI not only target specific viral strains but also have the power to predict future viral escapes. This ensures that the antibodies are designed in a way that it can provide protection against both current and future outbreak-causing viruses.

### Example 2

The efficacy of the immunogen peptides (lead candidates) described in Example 1 (Table 3) towards PRRSV neutralization will be evaluated through in vitro trials. Immunogen peptides designed from PRRSV epitopes will be synthesized using solid-phase peptide synthesis (SPPS) techniques. These peptides will be purified and validated before in vitro testing for their ability to stimulate immune cells and induce anti-PRRSV antibody production. Neutralization assays will evaluate the peptides' efficacy in preventing viral entry. The proliferation of specific antibodies will be monitored to assess immune response. In vivo characterization in swine will be conducted to confirm immunogenicity and protective efficacy.

Both the in vitro and in vivo tests will go through several rounds of design-build-test-learn iterations, helping to progressively gravitate towards the most successful designs. The machine learning pipeline described herein is capable of learning from both positive and negative results, thereby optimizing outcomes with each iteration.

### Example 3. Case Study with Infection Bronchitis Virus (IBV)

The Infectious Bronchitis Virus (IBV) is an avian coronavirus primarily responsible for causing infectious bronchitis in chicken. It can also circulate among other avian species. It was initially identified in North Dakota, USA and has since resulted in high morbidity and ranks just behind highly pathogenic avian influenza (HPAI) in its economic impact on the global poultry industry. The virus is responsible for a significant economic loss in poultry industries all over the world. IBV's ability to target both the upper respiratory and reproductive systems (with sporadic instances of nephritis) in chickens adds to the complexity and severity of pathogenesis. The genome of IBV consists of linear, single-stranded, positive-sense RNA, approximately 27-28 kb in length. This RNA encodes around 20 structural and nonstructural proteins. The 4 structural proteins, span functional members including the antigenic spike(S), membrane (M), envelope (E), and nucleocapsid (N) proteins. The antigenic spike protein facilitates attachment to host cells and mediates membrane fusion, thereby influencing viral tropism. In contrast, nucleocapsid proteins, encoded by the last open reading frame (ORF) of the genome, bind non-specifically to RNA and performs multiple functions essential for the virus's lifecycle. These proteins are vital for IBV's ability to infect and replicate in host cells. Furthermore, as an RNA virus, IBV exhibits a high mutation and recombination rate, frequently giving rise to new strains that challenge existing control measures. One of its subunits, S1, has higher amino acid variations ranging from 20% to 50% between IBV strains.

Overall, the error-prone viral polymerase and its recombination mechanisms lead to a highly diverse viral population with 9 known genotype variants and at least 30 serotypes. As a result, this diversity significantly complicates the surveillance and intervention strategies, including efforts to control spillover events, alluding to challenges in biosecurity and vaccination efforts.

IBV is considered a global pathogen, owing to its ability to swiftly spread through poultry populations. This rapid transmission is particularly evident in unvaccinated birds, which can shed the virus over prolonged periods, thereby promoting its spread within flocks. Therefore, controlling IBV is challenging owing to strict biosecurity measures and uniform flock ages. Nevertheless, vaccination remains essential for boosting birds' immune systems to manage IBV infections. Although numerous vaccines for IBV exist, only live attenuated and inactivated vaccines are commercially utilized. Notwithstanding, the kinetics of live attenuated IBV vaccine and the associated immune response in egg-laying hens are at best modestly understood to date. Additionally, commercial Modified Live Virus (MLV) vaccines for IBV pose risks, including reversion to virulence, recombination with circulating serotypes, and tissue damage in vaccinated birds.

To this end, immunoinformatics plays a pivotal role in identifying epitopes and designing vaccines for a range of critical diseases. Immunoinformatics has the capability to combine state-of-art computational optimization algorithms. It can reason over sequence and structural biological data from disease-causing viral variants to identify potential epitopes—i.e., specific parts of pathogenic proteins (antigens) that trigger the host immune response and are also under selection pressure to mutate to evade being neutralized by host immune proteins (antibodies). A carefully crafted immunoinformatics investigation not only enhances precision by unraveling a ranked list of most immunogenic and conserved regions of viruses, but also expedites vaccine design campaigns.

In this context, a multi-epitope vaccine (MEV) has emerged as a key therapeutic strategy. MEVs are peptide-based vaccines comprising T cell and B cell epitopes (i.e., multiple fragments of antigenic proteins strung together by linker amino acids), designed to elicit potent cellular and humoral immune responses. MEVs represent a promising approach for combating viral infections by eliciting a comprehensive immune response. These vaccines leverage T cell receptor (TCR) recognized Major Histocompatibility Complex (MHC)-restricted epitopes from several target antigens. MEVs also demonstrate enhanced immunogenicity because they pack in more than one viral antigenic epitope. Additionally, they provide long-lasting immune protection minimizing the side effects commonly associated with traditional vaccines. MEV peptides bear the potential to be highly effective prophylactic and therapeutic agents. However, identifying the most suitable target antigens and a rank ordered list of their immunodominant epitopes is crucial to design an effective MEV. Moreover, developing an appropriate delivery system is essential for the success of these vaccines. Therefore, the efficacy of MEV depends greatly on the selection of suitable candidate antigens and the affiliated immunodominant epitopes as the first step. Subsequently, the MEV must be designed for an optimal size, optimized for the biochemical objective of being a stable, high affinity binder to a known host protein to elicit the immune response. Ancillary design objectives include the right amount of aliphaticity, isoelectric point, extinction coefficient, half-life, and mean hydrophobicity.

To this end, demonstrated herein is a structurally and functionally-guided in silico, rank-ordered MEV library design strategy-PsiVax (Predictive Structural Immunoinformatics tool for Vaccine design) against chicken IBV. The conserved regions of nucleocapsid (N) and spike(S) proteins from 56 IBV genomes were prioritized, which are experimentally labeled for their potential ability to induce a strong immune response and span the known pathogenic variants.

While a set of primary MEVs can be generated by combinatoric piecing of existing viral epitope fragments held together by known experimentally optimized amino acid linkers, they do not guarantee the best possible binding and immunogenic properties unless they are affinity matured through directed evolution. This gap is bridged in PsiVax by integrating machine-learning-based protein diffusion model (RFDiffusion) to design an ensemble of MEVs which adhere to the secondary and tertiary structure of the top native MEV.

RFdiffusion is a machine-learning-based structural protein design tool and capable of designing ensembles of protein architectures, including monomers, shape-complementary binders, and symmetric oligomers—all of which can be leveraged for designing high-affinity peptide-based vaccines. RFdiffusion's ability to construct scaffolds that preserve the geometry of key structural motifs makes it highly valuable for our design objective in PsiVax. We leverage RFDiffusion within PsiVax to design libraries of MEVs capable of preserving the secondary structure and presenting multiple epitopes in a defined spatial configuration stably held in place by constant linker sequences. The sequence design step is accomplished by ProteinMPNN. These steps aid in generating protein sequences that fold into the desired MEV structure.

By integrating ProteinMPNN (and RFdiffusion) within PsiVax, we zero in on a tractable sequence space which is structurally stable. These designs are thereafter functionally profiled for nine factors-antigenicity, non-allergenicity, non-toxicity, interaction energy score with target protein, theoretical isoelectric point, extinction coefficient, predicted half-life, aliphatic index, and hydropathicity. Top designs are finally subjected to 20 ns all-atom molecular dynamics simulations (MD) for temporal fluctuation of receptor-binding energetics. Over time, MD simulations reveal critical insights into molecular processes that would be difficult or impossible to observe experimentally. MD simulations allow for an accurate representation of biomolecular interactions providing insights into the structure, dynamics, and function of these biomolecules at an atomic level. Since the last MD step is computationally demanding, the user can tailor the number of top designs they would like to assess. We have used (a default) six top designs to be evaluated their temporal binding energetics (stability) along with the parental MEVdesigned sequence.

The integration of RFdiffusion and ProteinMPNN in PsiVax enables the generation and screening of a putative multi-epitope vaccine (MEV) library, that adheres to a target scaffold that present multiple epitopes with high structural fidelity. This strategy paves the way for an innovative approach to structurally stable vaccine candidates. Incorporating multiple epitopes and preserving their native antigenic dihedrals, an MEV mimics multiple epitopes all within a single peptide, and consequently heightens immunogenicity. PsiVax is aimed at not only generating a comprehensive MEV design library but also significantly reduces the trial-and-error process in identifying lead vaccine candidates for experimental testing (i.e., streamlining vaccine biomanufacturing workflows) and adding structure-level interpretability to each designed MEV. Overall, PsiVax has the potential to design therapeutic interventions to control viral pathogens such as IBV, benefiting the poultry industry, animal health, and overall global agricultural economy. While demonstrated for IBV, this serves as an exemplar and not a research endpoint, as the PsiVax protocol is generalizable to design MEVs against any viral pathogen with the knowledge of viral genome(s), and a host receptor protein (See Example 1).

### Example 4. Results of Case Study with Infection Bronchitis Virus (IBV)

This example discusses the results of the case study describe in Example 3.

### IBV Viral Genomes and Proteomes

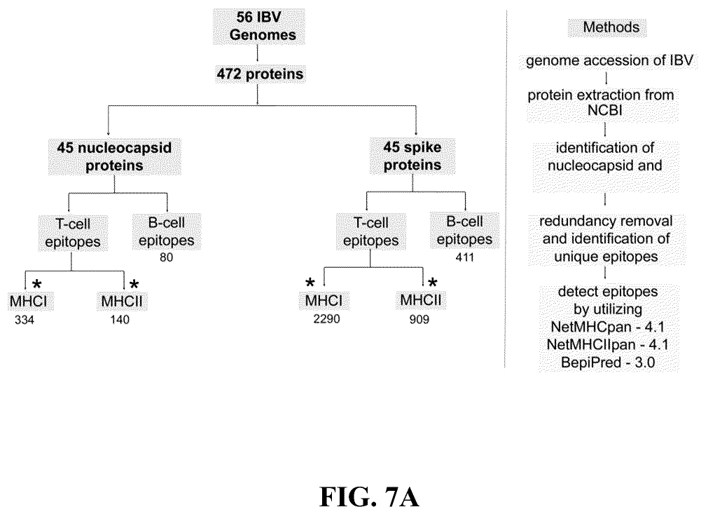

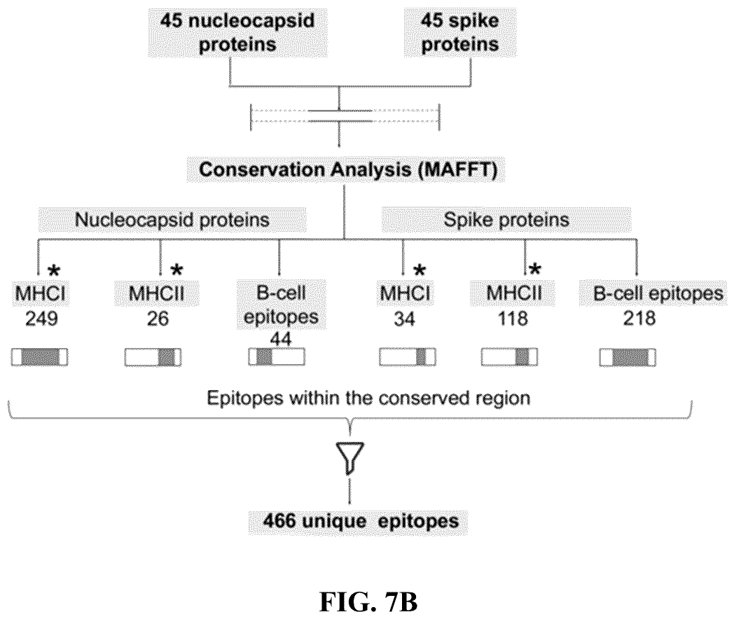

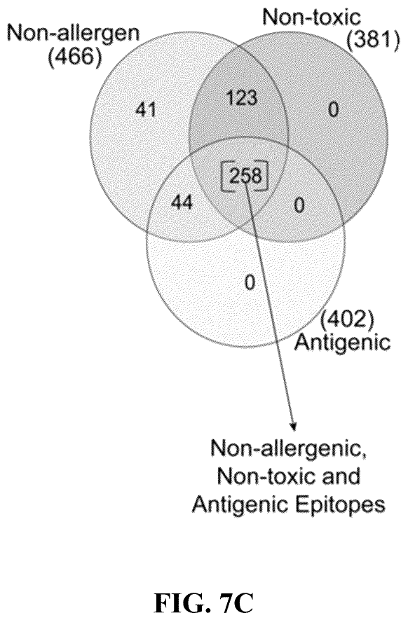

56 IBV genomes were analyzed to identify the complete proteome, resulting in the extraction of 472 associated proteins. From these, 90 proteins of particular interest were identified, which included 45 N proteins and 45 S proteins. These proteins were subsequently screened for epitope identification. This process is illustrated in FIG. 7A.

### Prediction of T and B-Cell Epitopes

For N proteins, 334 MHC-I epitopes were identified using NetMHCpan 4.1, 140 MHC-II T-cell epitopes using NetMHCIIpan 4.0, and 80 B-cell epitopes using BediPred 3.0. Similarly, for the spike proteins, 2,290 MHC-I epitopes, 909 MHC-II T-cell epitopes, and 411 B-cell epitopes (FIG. 7A) were identified. To ensure a comprehensive and unique dataset, redundant epitopes were eliminated, resulting in a non-redundant epitope dataset.

### Epitope Conservation Analysis

The analysis revealed a diverse set of 466 highly conserved epitopes across the 45 variants each of N and spike proteins. From the N protein variants, 249 MHC-I epitopes, 26 MHC-II epitopes, and 44 B-cell epitopes were identified. The spike protein variants yielded 34 MHC-I epitopes, 118 MHC-II epitopes, and 218 B-cell epitopes. In total, 466 unique epitopes demonstrated high conservation across the variants (FIG. 7B). These findings, based on the Epitope Conservancy Analysis using IEDB, provide a robust foundation for targeting conserved regions in IBV vaccine design.

### Allergenicity, Antigenicity, and Toxicity Analysis

Out of 466 epitopes, AllerCatPro 2.0 found all of them to be non-allergen. VaxiJen 2.0 predicted 302 out of 466 epitopes to be capable of inducing antigenic response. ToxinPred 3.0 found 381 out of 466 epitopes to be non-toxic. Subsequently, epitopes demonstrating antigenicity, non-allergenicity, and non-toxicity were selected. This screening resulted in the identification of 147 MHC-I epitopes, 87 MHC-II epitopes, and 25 B cell epitopes. In total, 258 unique epitopes were identified and selected for further analysis (FIG. 7C).

### MEV (SmallTope and BigTope) Structure Prediction and Properties

Both SmallTope and BigTope incorporate regions spanning MHC class I, MHC class II, and B-cell epitopes, along with linkers and adjuvants (Table 4), ensuring broad immunogenic potential. BigTope and Smalltope had several overlapping patches of epitopes, which included both MHC-I and MHC-II. The optimal size and weight of both constructs (177 residues/18.82 KDa for SmallTope, 394 residues/41.33 KDa for BigTope) suggest they are within a suitable range for vaccine candidates, balancing between being large enough to be immunogenic and small enough for efficient production and delivery (Table 5). Additionally, instability indices of 30.28 (SmallTope) and 22.91 (BigTope) fall below 40, indicating stable protein folds.

TABLE 4
SmallTope and BigTope Sequences

| | |
|---|---|
| SmallTope | MRIVYLLLPFILLLAQGAAGSSQALGRKSDCFRKSGFCAF |
| (SEQ ID NO: 62) | LKCPSLTLISGKCSRFYLCCKRIWGEAAAKGLLVLPPIITA |
| | AAYSVPDAWYFYYAAYERNNAQLEFGPGPGALTSDEER |
| | NNGPGPGGSGVPDNENGPGPRLSSLSVLAKKDVVTEQYR |
| | PPKKKLGGPKPPKVGSSG |
| BigTope | MRIVYLLLPFILLLAQGAAGSSQALGRKSDCFRKSGFCAF |
| (SEQ ID NO: 63) | LKCPSLTLISGKCSRFYLCCKRIWGEAAAKIPQNAPNGIVF |
| | AAYTYIKWPWYVWLAAYGLLVLPPIITAAAYKLGGPKPP |
| | KVAAYGRKPVPDAWYFYYAAYERNNAQLEFDDEPKVA |
| | AYRFSDGGPDGNFGPGPGRLSSLSVLASGPGPGLLVLPPII |
| | TGPGPGITAGDVVTLGPGPGRINHLGITQGPGPGNLTVTD |
| | EVIGPGPGLSILKTYIKGPGPGDFDFDDELSKGPGPGFEGS |
| | GVPDNENGPGPGVINWGDSALGGPGPGRAAKIIQDQKKG |
| | GPGPGRSTAASSAAKKDVVTEQYRPKKSYVYYYQSAFR |
| | KKKLGGPKPPKVGSSGKKKATKGKTDAPAKKRSNQGTR |
| | D |

TABLE 5
Physiochemical properties of the designed
SmallTope and BigTope vaccines and remarks.

| Parameter | SmallTope | BigTope | Functional Marker |
|---|---|---|---|
| Number of amino | 177 | 394 | Optimal size |
| acids | | | |
| Molecular weight | 18.82 KDa | 41.33 KDa | Optimal weight |
| Theoretical pI | 9.51 | 9.64 | Significantly basic |
| Extinction | 23,295 M⁻¹cm⁻¹ | 63175 M⁻¹cm⁻¹ | High light absorbance |
| coefficient | (assuming cysteines) | (assuming cysteines) | (necessary in protein |
| | 22,920 M⁻¹cm⁻¹ | 62800 M⁻¹cm⁻¹ | quantification and |
| | (assuming reduced | (assuming reduced | purity assessment) |
| | cysteines) | cysteines) | |
| Estimated half-life | 30 hours | 30 hours | Long half-life (higher |
| | (mammalian | (mammalian | the better) |
| | reticulocytes, | reticulocytes, | |
| | in vitro) >20 | in vitro) >20 | |
| | hours (yeast, | hours (yeast, | |
| | in vivo) >10 | in vivo) >10 | |
| | hours | hours | |
| | (Escherichia | (Escherichia | |
| | coli, in vivo) | coli, in vivo) | |
| Instability | 30.28 | 22.91 | Stable |
| index | | | |
| Aliphatic index | 80.00 | 73.12 | Thermostability (higher |
| | | | the better) |
| Grand average | −0.168 | −0.308 | Hydrophilic (lower the |
| of | | | more hydrophilic) |
| hydropathicity | | | |
| (GRAVY) | | | |

Next, Ramachandran plot analysis revealed distinct structural compositions. SmallTope is predominantly a-helical, featuring a short helix (Ile3-Leu6) and a longer a-helix (Leu7-Lys34). Beta-strands (e.g., Gly36-Phe40, Thr47-Ser54) form a compact sheet-like region, while tight turns (e.g., turn at Ala39-Lys42) create well-defined loops. BigTope exhibits a balanced a-helix and β-sheet composition, with stable a-helices (e.g., Ile3-Gly20, Ser22-Lys34) and β-strands (e.g., Gly36-Phe40, Thr47-Lys52) interspersed with tight turns (e.g., Ser54-Tyr57).

BigTope's structure is further stabilized by a salt bridge between Lys61 and Glu66, and an 11-residue leucine/isoleucine-rich hydrophobic core. Contact maps for both constructs reveal multiple long-range interactions, indicating structural coherence crucial for maintaining native folds and preserving immunologically relevant epitopes. These structural features collectively suggest that both SmallTope and BigTope possess the stability and conformational integrity necessary for effective vaccine candidates, potentially eliciting robust and diverse immune responses.

Docking Between SmallTope, BigTope and chTLR4

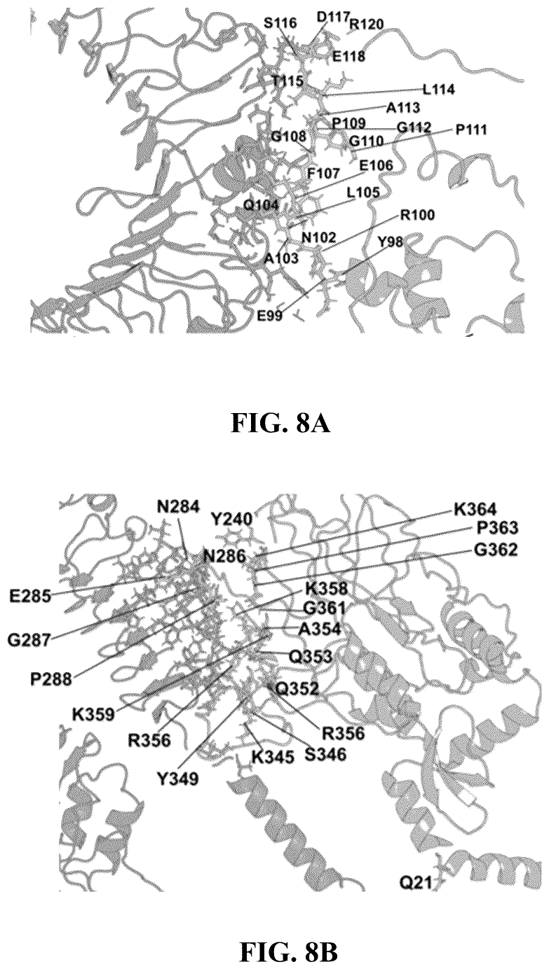

Molecular docking simulations revealed distinct binding interfaces between our Multi-Epitope Vaccines (MEVs) and chicken Toll-like Receptor 4 (chTLR4). HADDOCK 3.0 analysis yielded scores of −70.5 and −60.8 for SmallTope and BigTope respectively, indicating favorable interactions compared to random docking. These scores reflect a combination of energetic components, including van der Waals, electrostatic, and desolvation energies. Interacting residues in both MEVs originated from MHC-I, MHC-II, and B-cell epitope regions. BigTope engaged chTLR4 over a broader surface area compared to SmallTope's more linear interaction (FIGS. 8A and B). Notably, BigTope's AvBD region also participated in binding, consistent with its known role in immune response generation. Some interacting residues occupied flexible and outlier regions on the Ramachandran plot, suggesting potential functional significance. These findings highlight the structural basis for the MEVs' potential immunogenicity through chTLR4 engagement, known to induce protective immune responses in chickens.

### Design and Evaluation of MEV Variants for Optimization

We generated a diverse library of 1,000 MEV candidates using RFdiffusion and ProteinMPNN, comprising 500 variants each for SmallTope and BigTope. To ensure these variants were not random but aimed at improving the initial MEVs, we maintained the original interacting residues, adjuvants, and linkers. Initially, 50 protein backbones were generated with partial diffusion set at 20%, balancing sequence variation with structural integrity. Subsequently, ProteinMPNN was used to generate 10 sequences for each backbone, preserving conserved elements while exploring structural variations.

These variants were compared with the original designs, revealing potential interactions that were not identified previously in epitope regions with chTLR4. Analysis of the 1,000 variants identified numerous potential interactions within 6.5 Å across different epitope regions. SmallTope variants exhibited 303, 419, and 36 possible interactions in MHC-I, MHC-II, and B-cell epitope regions, respectively, while BigTope variants showed 866, 297, and 957 interactions in the corresponding regions. Overall, the BigTope constructs exhibited a substantial increase in the number of interactions within the B-cell epitope region. These variations highlight the structural flexibility in vaccine design, where previously non-interacting epitopes become interactive in variants, enhancing the robustness of our designs. Moreover, sequence similarity among SmallTope variants ranged from 56% to 70%, while BigTope variants spanned 48% to 52%. Notably, over half of the variants demonstrated higher binding energies than the original MEVs while maintaining similar secondary structural characteristics. We ranked variants based on their structural similarity to the originals and binding interaction energies from HADDOCK docking. The 50 variants, with the five best performers from each group, were selected for further molecular dynamics simulations in the next step. Overall, this comprehensive approach leverages advanced computational methods to generate and evaluate a rich set of MEV candidates, potentially improving upon the original designs while maintaining their core structural and functional properties.

### Binding Free Energy of Variant-chTLR4 Complexes

The binding free energy of the Variant-chTLR4 determines the stability and elucidates the conditions necessary for complex formation. The utilization of Poisson-Boltzman allows for an in-depth decomposition utilizing the thermodynamic cycle to extrapolate the significant components necessary for the binding process. The binding free energy for a complex can be estimated as follows:

$$Δ {G}_{bind} = 〈 {G}_{COM} 〉 - 〈 {G}_{REC} 〉 - 〈 {G}_{LIG} 〉 \qquad ( 1 )$$

Where each term on the right in Eq. (1) is given by

$$〈 {G}_{x} 〉 = 〈 {E}_{MM} 〉 + 〈 {G}_{sol} 〉 - 〈 TS 〉 \qquad ( 2 )$$

Thus, resulting in the Gibbs free energy equation upon binding, presented as

$$Δ {G}_{bind} = Δ H - T Δ S \qquad ( 3 )$$

The effective binding free energy is comprised of the enthalpy of binding ΔH and the conformational entropy after ligand binding-TAS 53. Eq. (3) can further be deconstructed into separate components where the enthalpy of binding is the sum of the molecular mechanical energy (ΔE_{MM}) in the gas phase and the solvation energy (ΔGₛₒₗ). Expressed as:

$$Δ H = Δ {E}_{MM} + Δ {G}_{sol} \qquad ( 4 )$$

Contributors to the mechanical energy are as followed:

$$Δ {E}_{MM} = Δ {E}_{bonded} + Δ {E}_{nonbonded} = ( Δ {E}_{bond} + Δ {E}_{angle} + Δ {E}_{dihedral} ) + ( Δ {E}_{ele} + Δ {E}_{VdW} ) \qquad ( 5 )$$

Where the Van der Waals (ΔE_{VdW}) and the electrostatic (ΔEₑₗₑ) interactions are accounted for their energetic contributions. Furthermore, the ΔE_{bonded} corresponds to the internal energy considering the motion of molecules, vibrational motion, and electric energy of the proteins. Entropic components are determined by the Interaction Entropy (IE) method. Additionally, the solvation energy is comprised of polar and nonpolar components, shown as

$$Δ {G}_{sol} = Δ {G}_{polar} + Δ {G}_{non - polar} \qquad ( 6 )$$

The polar component is estimated by the Poisson-Boltzmann model while the nonpolar component is proportional to the solvent accessible surface area (SASA). The SASA is calculated by the proportionality constant that is derived from experimental solvation energies of small nonpolar molecules. Thus, solvent contributions to the binding free energy are implicitly calculated by Poisson-Boltzmann.

Poisson-Boltzmann was performed on each of the Variant-chTLR4 complexes, where calculations were run for the duration of the 20 ns MD simulation to determine the binding affinity show in Table 6.

TABLE 6
Binding free energy of multitope variant
structure bound to chTLR4.

| | ΔG Binding Free |
|---|---|
| Variant | Energy (kcal/mol) |
| SmallTope | 135.26 ± 9.94 |
| Small-Var100 | 47.47 ± 17.48 |
| Small-Var150 | 43.19 ± 18.91 |
| Small-Var340 | 6.77 ± 19.63 |
| Small-Var347 | 233.62 ± 10.47 |
| Small-Var394 | 92.32 ± 13.42 |
| BigTope | 8.11 ± 31.87 |
| Big-Var22 | −9.53 ± 12.86 |
| Big-Var133 | −27.22 ± 17.03 |
| Big-Var142 | 51.33 ± 11.55 |
| Big-Var320 | 6.81 ± 17.16 |
| Big-Var410 | −29.82 ± 21.17 |

The binding free energy of the small variants Small-Var100, Small-Var150, and Small-Var340 suggest improvement in ligand binding in contrast to SmallTope. Small-Var340 has a significant improvement in binding, however, even with the advancement in protein-protein interaction, the

6.77±19.63 kcal/mol suggests a relatively weak binding process. Small-Var347 and Small-Var394 performed poorly conveying notable instability in the protein-protein interactions. Bigtope has a binding free energy of 8.11+31.87 kcal/mol insinuating a weaker binding process in comparison to its variant counterparts. There is a significant increase in favorable interactions in Big-Var133 and Big-Var410 demonstrating that the BigTope variants are stable binders to chTLR4. Big-Var142 expressed a thermodynamically unfavorable binding process due to restrictive residue interactions resulting in poor binding.

### Discussion

Multi-epitope vaccines (MEVs) have emerged as a promising strategy for developing effective and broad-spectrum immunizations against various pathogens. Our research advances this approach by incorporating a systematic immunoinformatics-driven methodology targeting Infectious Bronchitis Virus (IBV), a highly mutable coronavirus affecting the poultry industry.

To ensure cross-protection against multiple IBV variants, we conducted a rigorous selection of epitopes conserved in at least 80% of analyzed strains, aligning closely with successful vaccine strategies against other highly mutable viruses such as influenza and PRRSV.

A common challenge in protein-based vaccines is its instability and misfolding. To address this, we designed two optimized MEVs-SmallTope and BigTope. They included 23 of the highest conserved and overlapping epitopes from N and S proteins, with the addition of appropriate linkers and adjuvants. SmallTope's predominantly a-helical structure with compact β-sheets provides a stable core, while BigTope's balanced a-helix and β-sheet composition, along with its salt bridge (Lys61-Glu66) and hydrophobic core, suggest enhanced stability. The observed long-range interactions in both constructs indicate structural coherence, essential for preserving native folds and epitope conformation. While structural integrity is crucial for vaccine efficacy, it is equally important to assess the vaccine's ability to induce an immune response. For evaluating the immunogenic potential, we employed chicken Toll-like receptor 4 (chTLR4), a critical pattern recognition receptor known to initiate robust innate and adaptive immune responses through NF-κB signaling pathways. Molecular docking and subsequent dynamics simulations showed significant and stable interactions between chTLR4 and both vaccine constructs, underscoring their stability and capability to effectively engage immune activation mechanisms. Docking studies using HADDOCK3 revealed that SmallTope engages in 17 interactions with the MHC-I and MHC-II regions, whereas BigTope forms 15 interactions with the MHC-II and B-cell epitope regions (Supplementary Data). The AvBD region of BigTope is implicated in binding, a characteristic known to elicit an immune response.

Even with the successful design of two MEVs, we observed variations in the binding sites that underscore the impact of sequence restrictions on MEV performance. Therefore, our approach addresses a critical limitation in vaccine development—the inherent lack of variability and adaptability in traditional designs, which often rely on a “hit or miss” strategy due to limited opportunities for refinement. Initial docking analyses indicated that the B-cell regions for SmallTope and MHC-I regions for BigTope did not interact with chTLR4. To optimize our immunoinformatics-driven methodology, AI-based protein design tools-specifically RFdiffusion and ProteinMPNN-were employed. These tools enabled a systematic exploration of sequence variants that conserved key epitopes while enhancing receptor binding affinities. Many of the top-performing variants demonstrated improved docking scores relative to the parental constructs, indicative of enhanced binding energetics to chTLR4. In certain instances, epitopes that were previously peripheral to the receptor interface established contacts, underscoring how targeted backbone manipulation can expose cryptic or partially buried epitopes. Consequently, from a pool of 1000 variants, we selected 10 promising SmallTope and BigTope variants based on docking score and conducted detailed MD simulations to evaluate their performance and interactions with chTLR4.

Variants with stronger receptor affinity exhibited lower (more negative) binding free energy, with Big-Var133 (−27.22 kcal/mol) and Big-Var410 (−29.82 kcal/mol) demonstrating the strongest binding. Big-Var133's stability was driven by an extensive hydrogen bonding network including R120 and E285, reinforcing receptor engagement. P339 (−3.39 kcal/mol), P121 (−3.19 kcal/mol), V330 (−2.93 kcal/mol) and Y98 (−2.33 kcal/mol) reinforce the hydrophobic core, contributing to compact folding and preventing solvent exposure, which in turn strengthens receptor binding. Also, well-positioned K229-E285 salt bridge played a crucial role in stabilizing its interaction.

In contrast, Big-Var410, despite its strong binding, formed few hydrogen bonds, suggesting that its stability relied primarily on hydrophobic interactions rather than electrostatics. Additionally, Big-Var410 despite having the lowest binding energy (−29.82 kcal/mol), lacked salt bridges, preventing optimal electrostatic stabilization. P126 (−2.51 kcal/mol), W64 (−2.47 kcal/mol), Y84 (−2.07 kcal/mol), form strong hydrophobic interactions that compensate for limited hydrogen bonds and salt bridges, contributing to the low overall ΔG. Hydrophobic contacts A123, A124, Y125, P126, L46, 163, L103 and L393 created a stable hydrophobic core, preventing excessive solvent exposure. This compensated for the absence of salt bridges, keeping the BigTope structure compact and allowing effective receptor engagement. This highlights the importance of hydrophobic stabilization in multi-epitope vaccine design, even in cases where electrostatic interactions are weak.

Big-Var22 shows a negative binding free energy (−9.53 kcal/mol) that is supported by a strong hydrogen bonding network. It also participates in inter-chain polar and hydrogen bonding, along with several hydrophobic interactions that contribute to overall compactness and receptor binding stability such as polar interaction network involving L25, S29, F32, R33 and S115. The presence of these strong interactions explains the relatively stable binding. Var22 has weaker stabilizing residues, fewer strong interchain interactions, and several destabilizing contributions from residues with positive energy values. These differences collectively explain the higher stability and more negative ΔG of Var133. In contrast, key residues in Big-Var133, such as R120 and P339, provide highly stabilizing electrostatic and hydrophobic interactions. Additionally, Big-Var133 benefits from multiple proline residues that enhance structural rigidity.

Original BigTope and Big-Var320 exhibit moderately positive binding free energies (6.81 kcal/mol). For Big-Var320, weak inter-chain stabilizations such as the limited hydrogen bonding and lack of robust salt bridges-alongside insufficient hydrophobic contributions, result in reduced binding efficiency. Big-Var142 demonstrates extreme instability (51.33 kcal/mol), driven by a complete absence of salt bridges, only limited inter-chain hydrogen bonds, and disruptive steric clashes, compounded by inadequate hydrophobic stabilization. This highlights how deficiencies in electrostatic and structural complementarity critically undermine binding.

Of the SmallTope variants, it was observed that Small-Var340 showed the most promise in improving the binding affinity of SmallTope-chTLR4 complexes, while in contrast, Small-Var347 inherently deteriorated the binding process. Salt bridges play a crucial role in protein-protein interactions, contributing significantly to the stability of specificity of these interactions. They can facilitate the formation of stable complexes by thermodynamically contributing with their favorable electrostatic attractions, offsetting the entropic cost of binding. From this point forward, one letter residue codes will be associated with residues for chTLR4 and three letter residue codes will be for SmallTope and its variants. Small-Var340 has a key salt bridge between Lys93-D309 located in the second section of the MHC-I domain. Of all the small variants, only Small-Var150 and Small-Var340 have a lysine in this position and only Small-Var340 activates its lysine for the formation of a salt bridge. This salt bridge is located at the end of the alpha helix of the second section of MHC-I contributing-2.763 kcal/mol to the binding free energy. Small-Var340 is the only one that has a salt bridge in the MHC-I domain whereas Small-Var100 has no salt bridges, Small-Var150 has a salt bridge in the B-cell domain, and SmallTope, Small-Var347, and Small-Var394 have a salt bridge in the MHC-II domain. In addition, Small-Var340 is unique among all small epitopes due to its full favorable contribution of MHC-I domain in the binding process whereas others do not have any interaction with chTLR4 until the third section of the MHC-I domain after the second AAY linker, where a majority of the MHC-I is isolated from the binding site. Furthermore, the docking orientation of Small-Var340, in comparison to its counterparts, encourages the contribution of MHC-I resulting in a primarily MHC-I domain driven binding process stabilized by Lys93. This salt bridge allows for the establishment of a hydrophobic core shaped by section two and section three of the MHC-I including an important network of hydrogen interactions involving multiple leucine and proline residues within section one further stabilizing the MHC-I. The location specificity of Lys93 in conjunction with the docking orientation of the Small-Var340 to chTLR4 reveals its importance in the stability of the binding process. For Small-Var340, the B-cell domain is completely secluded from the binding site and plays little part in the complex formation. What makes section one of MHC-II unique for Small-Var340 is that it is largely made up of glycine residues with half of the residues in the domain being glycine. This flexibility in section 1 allows for a hydrophobic interaction between Ala119-Met128 and the potential for cation-pi interaction between K229-Trp131. MHC-II had some favorable involvement as only the first and second section of MHC-II had any interaction with chTLR4. In the MHC-II domain, key hydrogen bonds between Q223 (−2.060 kcal/mol) and Gly118 (−1.065 kcal/mol), Q228 (−1.863 kcal/mol) and Pro102 (kcal/mol), and an intricate network between Q286 (−5.399 kcal/mol) with Glu88, Gly105, and Leu106 (3.000, −1.266, and −0.335 kcal/mol). From energetic analysis of the per residue contribution to the binding free energy, it was observed that for the Small-Var340 chTLR4 complex, there was a significant role that the chTLR4 glutamine seems to participate in for the complex formation. These crucial glutamines (Q223, Q252, Q286, Q335, and Q288) directly face the MHC-I and MHC-II, thus, allowing for the possibility of stabilizing hydrogen bonding as the proteins interact. The Small-Var340 complex has the most involvement of these glutamine residues alluding to the significant role they play in the binding process.

Conversely, Small-Var347 had the most unfavorable protein-protein interaction with staggering exacerbated results in comparison to the adverse results of the SmallTope. What makes Small-Var347 distinct relative to its variant counterparts, is that Small-Var347 has the lowest chain to chain interactions with chTLR4 with only three total hydrogen bonds (D309-Arg150 and F333-Lys102) and one salt bridge (D309-Arg150) located in the MHC-II domain. In this case, although the salt bridge was a particularly favorable event, it was not enough. Even with the second highest binding score from HADDOCK, this binding process was largely disadvantageous as the binding orientation resulted in binding driven by the B-cell domain. Although B-cell epitopes are meant for adaptive immune response, it could be that the linearity of the domain was not enough to securely bind to chTLR4. There were three critical favorable energetic contributions in the B-cell domain from Arg150 (−4.440 kcal/mol), Lys151 (−4.017 kcal/mol), and Leu169 (−1.988 kcal/mol). Outside of these three residues, all other residues in the B-cell domain had negligible or positive energetic contributions with very little hydrophobic interactions and minimal intra-chain hydrogen bonding suggesting an incredibly high conformational entropy and little stability in complex formation. In addition, the glutamine residues in chTLR4 played an inconsequential role in the binding to Small-Var347. The disparity between Small-Var340 and Small-Var347 can be attributed to the binding configuration of Small-Var347 on chTLR4 whereas Small-Var340 was largely driven by MHC-I and MHC-II, Small-Var347 was the complete opposite as it had little support from MHC-II and was dominated by the B-cell domain. Thus, the binding process was facilitated by two small linear portions of the B-cell domain providing limited surface area for the proteins to bind effectively.

Similar to Small-Var347, SmallTopes binding arrangement was also facilitated by a linear chain of residues. However, unlike Small-Var347, SmallTopes protein-protein interactions were based only in the third section of MHC-I, the GPGPG linker, and the first section of MHC-II resulting in more hydrogen bonding and the formation of a salt bridge. SmallTope had six total hydrogen bonds between SmallTope and chTLR4: K196-Glu118 (−1.758−0.143 kcal/mol), Q223 & S224-Ser116 (−0.112 & −0.237-−0.855 kcal/mol), C285-Phe107 (0.067-−2.608 kcal/mol), N313-Leu105 (−0.008-−0.923 kcal/mol), and Q335-Gln104 (−0.562-−0.821 kcal/mol) with the first two hydrogen bonds located in the MHC-II and the rest from MHC-I. Although a salt bridge does form between K196-Glu118, the presence of this interaction seems to be significant as the GPGPG linker seems to being to pull away from the bind site after MHC-I, the salt bridge seems to anchor the first section of MHC-II allowing for more interaction with MHC-II. On the other side, section three of MHC-I is anchored via hydrophobic interactions from M331 (−1.622 kcal/mol), F333 (−3.651 kcal/mol), Ala103 (−0.945 kcal/mol), and Leu105 (−0.923 kcal/mol) thus assisting in the orientation of the linear chain.

Small-Var100 and Small-Var150 have comparable binding free energies but, had different binding profiles while Small-Var394 had a non-comparable binding free energy but a similar binding profile to Small-Var150. Small-Var100 was bound by the AAY linker, section three of MHC-I, GPGPG linker, and section one of MHC-II. Small-Var150 and Small-Var394 were bound by section three of MHC-I, GPGPG linker, sections one and three of MHC-II, GPGPG linker, and section one from B-Cell. Small-Var100 had a total of eleven inter-chain hydrogen bonds with the distribution being four from MHC-I and seven from MHC-II. While on the other hand, Small-Var150 had a total of seven inter-chain hydrogen bonds where one comes from the GPGPG linker, two from section 1 of MHC-II, three from section three of MHC-II, and one from B-cell with the addition of a salt bridge between D301-Lys154 (0.154-−0.988 kcal/mol) in the B-cell domain. With the largest distinction between Small-Var100 and Small-Var150 being its binding orientation, Small-Var150 had a slightly better overall binding free energy which could be attributed to the capacity of glutamine interactions from chTLR4. For the Small-Var100 complex there are four glutamine residues in the binding site from chTLR4 that interact with Small-Var100: Q252, Q258, Q286, and Q288 (0.203, −0.433, 1.000, and −0.774 kcal/mol). Conversely, the Small-Var150 complex has the following glutamine residues from chTLR4: Q223, Q258, Q277, Q286, and Q288 (−1.624, −0.350, −0.148, −0.601, and −0.496 kcal/mol). Small-Var150, Small-Var394 has ten hydrogen bonds with four in section three of MHC-I, Pro111 and Gly112 from the GPGPG linker, one from section one of MHC-II, two from section three of MHC-II, and one from B-cell.

There are three key inter-chain networks of hydrogen interactions with K229-Gly103, Gly104, and Ile105 (−0.186-−1.548, −0.047, −0.851 kcal/mol), Q286-Thr142 & Gly141 (−4.002-−3.223 &−2.114 kcal/mol), and N313-Pro111 & Gly112 (−3.005-−6.041 &−1.692 kcal/mol). These hydrogen interactions largely drive the binding process with Pro111, Gly112, and Gly141 coming from the GPGPG linkers. Glutamine residues from chTLR4 are as follows: Q223, Q252, Q258, Q286, Q288, and Q335 (−0.969, 0.017, −0.703, −4.00, −2.01, and −1.10 kcal/mol). This observation further stipulates the necessity of the chTLR4 glutamine residues in the binding process for small epitopes. Though Small-Var394 arguably had the most favorable per residue energetic contributions, the conformation entropy was incredibly high, comparable to SmallTope (Supplementary FIG. 4) which could be due to the binding process being driven mostly by the first and third GPGPG linkers and a few adjacent residues to those linkers.

A commonality between SmallTope and most of its variant, with the exception of Small-Var100, there a is a shared favorable residue, Pro109 that is part of the first GPGPG linker that connects MHC-I and MHC-II. Another shared characteristic is that a majority of negative energetic contributors are located between section two of MHC-I and section one of MHC-II. Furthermore, what makes SmallTope and its variants distinct from BigTope and its variants is that region where the small epitopes bind do not vary by much, they are all relatively in the same area in comparison to the big epitopes where the range of binding varies substantially.

Overall, these findings underscore the importance of targeting highly conserved epitopes across diverse IBV strains, the role of structural stability in vaccine design, and leveraging AI-driven protein design to refine and expand the repertoire of MEV candidates.

### Methods

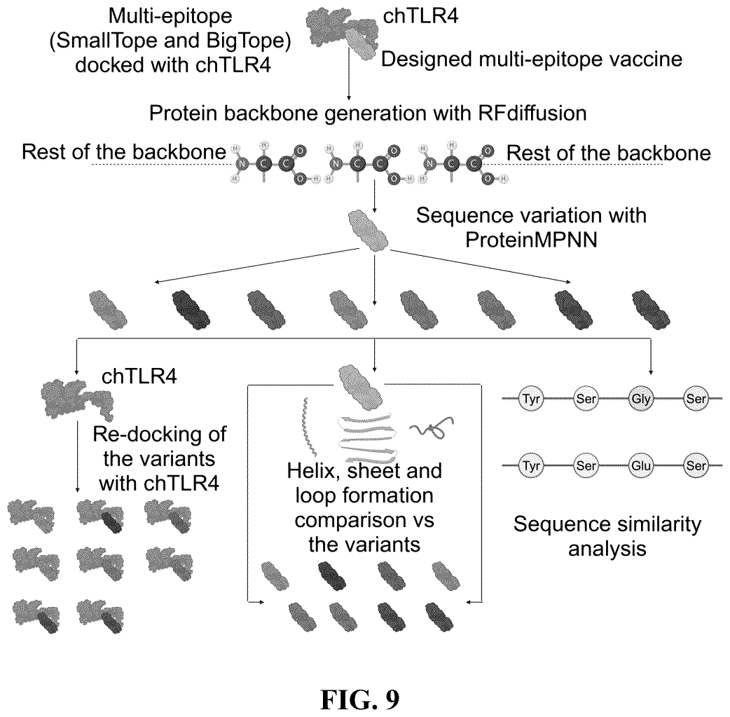

The methodology in this study encompasses the selection of epitopes for the vaccine, as illustrated in FIGS. 7A-C. It is followed by the construction, quality assessment, and biochemical evaluation of the MEV. Pre-trained deep learning (i.e., ProteinMPNN, RFdiffusion, ESMFold, AlphaFold) is used to preserve highly conserved epitopes within our designed MEV using a zero-shot approach (FIG. 9).

### Retrieving Viral Genomes and Proteomes

To identify epitopes in Infectious Bronchitis Virus (IBV), we accessed 56 IBV genomes from the NCBI database. Nucleocapsid (N) and spike(S) proteins are critical for viral attachment, entry, replication, and overall infection and account for 19% of all 472 annotated proteins. We isolated all variants of these N and S proteins.

### Prediction of T-Cell Epitopes

We utilized NetMHCpan 4.1 for MHC class I (MHC-I) T-cell epitope prediction and NetMHCIIpan 4.0 for MHC class II (MHC-II) T-cell epitope prediction. Both methods utilize a single allele framework that can integrate binding affinity and mass spectrometry (MS) data from public domain for model training. This methodology holds state-of-the-art performance along with boosted predictive power.

For NetMHCpan, we included all supertype Human Leukocyte Antigen (HLA) representatives in our epitope search. We adhered to the default parameters, setting the threshold for strong binders at the top 0.5% rank and for weak binders at the top 2% rank. Our analysis focused exclusively on the strong binding epitopes. In NetMHCIIpan 4.0, we selected a range of Human Leukocyte Antigen-DR Beta (HLA-DRB), HLA-DP, and HLA-DQ alleles for epitope prediction. The default criteria were applied, with a top 1% rank threshold for strong binders and a top 5% rank threshold for weak binders. Consistent with our approach in NetMHCpan, we focused solely on the strong binders in our study. This methodological rigor ensured that only the most promising epitopes were considered for further analysis.

### Prediction of B-Cell Epitopes

We used BepiPred-3.0 for B-cell epitopes prediction. BepiPred-3.0 uses protein language models to accurately predict linear and conformational B-cell epitopes. Top epitope percentage cutoff was set at higher confidence, which was the top 20% of the epitopes. As a default criterion, threshold for predicting B-cell epitope residues was set at 0.151 on a scale ranging from 0 to 1. Additionally, we considered only the linear epitopes for further analysis.

### Conservancy of the Sequences Analysis

We employed MAFFT (Multiple Alignment using Fast Fourier Transform) to perform sequence alignment and conservancy analysis of the N and spike proteins. Subsequently, we utilized the IEDB Epitope Analysis Toolkit for a comprehensive examination of epitopes within these aligned sequences. We identified and selected highly conserved protein regions for in-depth analysis. Epitopes present in at least 80% of the 45 N and 45 S protein sequences were chosen, with most residues exceeding 90% conservation. The only exceptions were two B-epitopes for spike proteins, which showed lower conservation across the 45 sequences (84.44% and 55.56% respectively). This curation process allowed us to prioritize epitopes with the highest conservation scores that significantly overlapped with conserved stretches. Our approach yielded a robust set of epitopes present across major IBV variants, providing broad coverage against this highly mutable virus.

### Allergenicity, Antigenicity, and Toxicity Analysis

To assess the allergenicity potential of the identified epitopes, we utilized AllerCatPro 2.0. AllerCatPro 2.0 is an advanced tool that predicts protein allergenicity and cross-reactivity potential, incorporating diverse protein characteristics and clinical relevance. We excluded all epitopes demonstrating either strong or weak evidence of allergenic response. The antigenicity-inducing capability of the epitopes was predicted using the VaxiJen 2.0 server. VaxiJen 2.0 is a gold standard tool for predicting viral immunogenicity. With the target set to virus, default threshold was set at 0.4 because it shows the highest accuracy in the model. Next, ToxinPred

3.0 was utilized for the prediction of potential toxicity of the epitopes. ToxinPred3.0 utilizes a large dataset of experimentally validated toxic and non-toxic peptides to predict peptide toxicity with higher accuracy over its predecessors. For this analysis, the hybrid ET+MERCI machine learning model was applied with a default threshold of 0.38. The default threshold was set below the toxic peptides' cutoff (0.5) to ensure a conservative approach and minimize the inclusion of any potentially deleterious sequence. Ultimately, we selected only those epitopes that exhibited no allergenic response, high induced antigenicity, and no toxicity all at once (FIG. 7C).

### Construction of the SmallTope and BigTope Vaccine

From the selected 258 epitopes, we chose only 23 with the highest conservation and overlapping regions to maintain the multi-epitope size within acceptable limits, thereby enhancing the potential for immunogenicity. In constructing the multi-epitope vaccine, we combined two different approaches. We constructed SmallTope and BigTope as our multi-epitope vaccines. SmallTope only contains highest conserved regions in all 45 S and 45 N proteins and holds eight epitope fragments. These epitopes target MHC class I, MHC class II, and B cell receptors. They were stitched using linkers to enhance immunogenic efficacy and mitigating the risk of creating junctional epitopes with low accessibility and enhance the presentation of the targeted epitopes.

On the other hand, BigTope was created to encompass about 3-fold larger segment consisting of 23 epitopes. The epitope composition is summarized in Table 7.

TABLE 7
Epitope Composition in SmallTope and BigTope MEVs.

| Vaccine | MHCI | MHCII | B Cell | N | Spike | Immunogenicity |
|---|---|---|---|---|---|---|
| Type | Epitopes | Epitopes | Epitopes | Proteins | Proteins | Enhancer |
| SmallTope | 3 | 3 | 2 | 5 | 3 | Avian beta- |
| BigTope | 7 | 11 | 5 | 11 | 12 | Defensin with |
| | | | | | | EAAAK |

We incorporated identical linkers and adjuvants while developing the SmallTope and BigTope. For the MHC class I epitopes, AAY linkers were utilized, while the MHC class II and B cell epitopes were connected via GPGPG and KK linkers, respectively. Furthermore, to enhance the vaccine's immunogenicity, a 65-amino acid avian beta-defensin (AvBD) sequence (GenBank accession NP_990324) was linked to the N-terminus with the presence of an EAAAK linker.

### 3D Structure, Physiochemical Properties and Ramachandran Plot Evaluation

The 3D architecture of the SmallTope and BigTope constructs were predicted using the latest version of AlphaFold (AlphaFold3). AlphaFold3 expands on its predecessor by accurately predicting interactions between biomolecules, marking a significant advance in computational structural biology. To explore the physicochemical properties of the multiepitope protein, we employed ProtParam. ProtParam allows the analysis of distinct physical and chemical parameters of any protein. Subsequently, a Ramachandran plot was generated using RAMAplot to assess the conformational angles of the amino acids within the protein.

Docking SmallTope and BigTope with chTLR4

Chicken Toll-like receptor 4 (chTLR4) plays a crucial role in initiating both innate and adaptive immune responses. Moreover, Studies focusing on single nucleotide polymorphisms (snps) have identified specific residues critical for chTLR4's immunogenicity (55). In our study, we predicted the chTLR4 structure using AlphaFold3 and docked it with our designed SmallTope and BigTope. Haddock2.4 web server was used for docking. DeepTMHMM, Phobius, and TopCon were employed to predict extracellular regions of chTLR4. DeepTMHMM model leverages a pre-trained language model to encode amino acid sequences and employs a state space model for topology decoding. This approach enables accurate prediction of membrane protein topology for both alpha-helical and beta-barrel structures. Similarly, Phobius is used for transmembrane toplogy and signal peptides prediction of a protein. Next, TOPCONS is a state-of-the-art tool that is widely used for consensus prediction of membrane protein topology. Within the identified extracellular domain, one residue was randomly set as active for docking, while the rest of the domain was designated as passive. This method produced a more stable docked complex compared to conventional random docking, suggesting it as a more effective model for examining chTLR4 interactions.

### Utilizing RFdiffusion and ProteinMPNN to Create Sequence Variants

Diffusion-model based RFdiffusion utilized a class of generative machine learning models to create the backbone of the docked SmallTope and BigTope. In our design process, we preserved the conserved linkers and adjuvants. Interacting residues (identified using a 6.5 Å cut-off from initial docking studies) were excluded from the diffusion process. We designated the interacting residues in chTLR4 as hot-spots for RFDiffusion to ensure that the generated structures would maintain interactions with chTLR4 similar to the original design. Using these parameters, we created 50 backbones with partial diffusion set at 20. Next, to optimize the SmallTope and BigTope designs, we employed ProteinMPNN, a deep learning-based protein sequence design method, to generate 10 sequences for each backbone. ProteinMPNN offers superior sequence recovery and structural robustness compared to traditional methods. This resulted in 500 sequence variants for each vaccine construct. We set the temperature parameter to 0.25 on a scale of 0-1, balancing sequence diversity with structural constraints. All other ProteinMPNN parameters were kept at their default values.

Prediction of the Structure Database and Re-Docking with chTLR4

ESMFold efficiently generated structures for the variants predicted by RFdiffusion and ProteinMPNN. We chose ESMFold for its ability to rapidly predict a large number of variant structures (1000) with accuracy comparable to AlphaFold. Subsequently, we employed HADDOCK3.0, an advanced integrative modeling platform for biomolecular interactions, to dock 500 SmallTope and 500 BigTope variants with chTLR4. To manage the high number of complexes, we utilized haddock-runner for batch processing. Interacting residues identified from initial docking studies were designated as active residues in HADDOCK3.0. This approach promotes binding interactions involving these specific residues with chTLR4 while allowing flexibility for interactions with other residues. Next, we applied the haddock-restrain script to generate appropriate restraints for each docking scenario, ensuring a balanced exploration of the binding interface.

### DSSP Based Structural Similarity and Sequence Similarity Analysis

The Dictionary of Secondary Structure of Proteins (DSSP) is a widely used algorithm and database that we used for standardizing the assignment of secondary structures for our MEV and its variation atlas. We calculated the percentage of alpha helices, beta sheets, and loop in each of the structures. Additionally, the overall similarity between the sequences was calculated.

### Assessment of the Quality of the Designed Variants

We generated 1000 variants each for SmallTope and BigTope, creating a comprehensive atlas for comparison against the original designs. This extensive collection allowed for a thorough exploration of potential improvements. The comparison was conducted across multiple criteria, with the most critical being the HADDOCK3.0 binding interaction score, which served as an in silico proxy of interaction strength. In addition to binding affinity, we assessed the secondary structure similarities between the atlas variants and the MEV to evaluate structural consistency. Lastly, sequence similarity analysis was performed to determine how sequence variation could still preserve the structural integrity critical for vaccine efficacy. Finally, the top ranked variants were subjected to molecular dynamics (MD) simulations for further analysis.

### Molecular Dynamics Simulations of Muli-Epitope-chLTR4 Complexes

A series of MD simulations were performed on the docked variants of the SmallTope and BigTopes via HADDOCK3.0 to chTLR4 with top ranked variants. MD simulations were performed with the GROMACS 2023 software and the AMBER99SB-ILDN forcefield. The Variant-chTLR4 complexes were placed a 10Å distance from the complex and the box edge, ensuring enough space for solvation and periodic boundary conditions. Complexes were then solvated with TIP3P water molecules and if necessary, chloride or sodium ions were added to neutralize the total system charge. The system was then further processed in a two-step final equilibration. The first step was to apply a constant number, volume, and temperature (NVT) simulation using the V-rescale algorithm without any position restraints for 100 ps. The second step was conducted at a constant number, pressure, and temperature (NPT) for a 100 ps simulation using the Parrinello-Rahman algorithm at 1 bar pressure and the V-rescale algorithm at 300K. In the production run, we utilized the leap-frog integrator to run a 20 ns simulation and an integration timestep of 2.0 fs. Periodic boundary conditions were applied in all directions throughout the simulations. The LINCS algorithm was used only to constrain the bonds involving hydrogen atoms. Electrostatic interactions were calculated using the Particle Mesh Ewald algorithm with cubic interpolation and a fast Fourier Transform set to 0.16 nm. The trajectories were saved every 10 ps during each simulation for analysis.

What is claimed is:

1. A method for identifying viral epitopes and/or paratopes, comprising:
identifying one or more viral proteins comprising one or more transmembrane viral proteins;
identifying one or more host receptor proteins comprising one or more transmembrane host proteins;
predicting the three dimensional structure of the one or more transmembrane viral proteins and the one or more transmembrane host receptor proteins;
predicting protein-protein docking poses of the of the one or more transmembrane viral proteins to the one or more transmembrane host receptor proteins to identify one or more viral epitopes and/or one or more host receptor paratopes.

2. The method of claim 1, wherein the step of identifying the one or more viral proteins comprising one or more transmembrane viral proteins and the step of identifying one or more host receptor proteins comprising one or more transmembrane host proteins comprises performing subcellular localization on the identified one or more viral proteins or the one or more host receptor proteins.

3. The method of claim 1, further comprising screening the one or more viral epitopes for allergenicity, toxicity, and antigenicity.

4. The method of claim 1, further comprising constructing an immunogen based upon the identified one or more viral epitopes.

5. The method of claim 4, wherein the construction step is performed using RFdiffusion and/or ProteinMPNN.

6. The method of claim 4, wherein the immunogen is a multi-epitope vaccine (MEV) comprising at least one T cell epitope and at least one B cell epitope.

7. The method of claim 6, further comprising generating a database of MEVs.

8. The method of claim 2, wherein the performing subcellular localization step is performed using BUSCA, Deep TMHMM, and/or DeepLoc.

9. The method of claim 1, wherein the predicting the three dimensional structure step is performed using ESMfold and/or AlphaFold2.

10. The method of claim 1, wherein the predicting protein-protein docking step is performed using HADDOCK 2.4, ClusPro 2.0, and/or GRAMM: Docking.

11. The method of claim 1, wherein the identification of the one or more transmembrane viral proteins and the one or more transmembrane host receptor proteins comprises selecting against isoforms and/or unstructured proteins.

12. The method of claim 1, further comprising generating a database of identified viral epitopes.

13. The database of identified viral epitopes generated by the method of claim 12.

14. The method of claim 1, wherein the viral proteins are porcine reproductive and respiratory syndrome virus (PRRSV) proteins.

15. The method of claim 1, wherein the viral proteins are Infectious Bronchitis Virus (IBV) proteins.

16. A computerized method for designing viral antibodies, comprising:
constructing an immunogen comprising at least one viral epitope from the database of identified viral epitopes of claim 13;
using artificial intelligence to design an antibody reactive against the immunogen.

17. The method of claim 16, further comprising screening the immunogen for solvent accessibility and selecting a stable immunogen prior to using artificial intelligence to design the antibody.

18. The method of claim 16, further comprising calculating the binding affinity of the antibody.

19. An immunogenic composition comprising:
at least one antigenic fragment of a porcine reproductive and respiratory syndrome virus (PRRSV) protein, wherein the protein is E envelope protein, GP2, GP3, GP4, GP5, membrane protein M, nsp3, nsp5, and/or ORF5a; and
a pharmaceutically acceptable carrier.

20. The immunogenic composition of claim 19, wherein the antigenic fragment comprises a polypeptide of any one of SEQ ID NOs: 1-61, or a functional variant thereof.

21. The immunogenic composition of claim 19, wherein the composition comprises more than one antigenic fragment.

22. The immunogenic composition of claim 19, wherein the antigen is a recombinant antigen.

23. The immunogenic composition of claim 19, wherein the pharmaceutically acceptable carrier comprises a diluent, adjuvant, antimicrobial agent, preservative, inactivating agent, or combinations thereof.

24. A method of immunizing and/or treating a subject against PRRSV comprising administering the composition of claim 19.

25. An antibody or an antigen binding fragment thereof capable of binding to an antigenic fragment of a porcine reproductive and respiratory syndrome virus (PRRSV) protein, wherein the protein is E envelope protein, GP2, GP3, GP4, GP5, membrane protein M, nsp3, nsp5, and/or ORF5a.

26. The antibody or antigen binding fragment thereof of claim 25, wherein the antigenic fragment of a PRRSV protein comprises a polypeptide of any one of SEQ ID NOs: 1-61, or a functional variant thereof.

27. A composition comprising the antibody or antigen binding fragment thereof of claim 19.

28. A multi-epitope vaccine (MEV) comprising:
at least two antigenic fragments of an Infectious Bronchitis Virus (IBV) protein, wherein the antigenic fragments comprise at least one B-cell epitope and at least one T-cell epitope.

29. The MEV of claim 28, wherein the MEV comprises an amino acid sequence having at least 90% sequence identity to SEQ ID NO: 62 or 63.

30. A composition comprising the MEV of claim 28.

31. The composition of claim 30, further comprising a diluent, adjuvant, antimicrobial agent, preservative, inactivating agent, or combinations thereof.

32. A method of immunizing and/or treating a subject against IBV comprising administering the composition of claim 30.


SEQUENCE LISTING (63 sequences)

SEQ ID NO: 1 - protein, 11 aa, synthetic construct
PHLMAALHVAC

SEQ ID NO: 2 - protein, 11 aa, synthetic construct
ANFLWMLSRSS

SEQ ID NO: 3 - protein, 11 aa, synthetic construct
VAASCTLFVVL

SEQ ID NO: 4 - protein, 10 aa, synthetic construct
MAHQCACFHF

SEQ ID NO: 5 - protein, 11 aa, synthetic construct
HFQSTNKVALT

SEQ ID NO: 6 - protein, 11 aa, synthetic construct
FFLLVGAQHIM

SEQ ID NO: 7 - protein, 11 aa, synthetic construct
ANFLWMLSRSS

SEQ ID NO: 8 - protein, 10 aa, synthetic construct
MAHQCACFHF

SEQ ID NO: 9 - protein, 11 aa, synthetic construct
AIILYLFKYRG

SEQ ID NO: 10 - protein, 11 aa, synthetic construct
HQIDGGNWFHL

SEQ ID NO: 11 - protein, 14 aa, synthetic construct
VSACVNFTDYVAHV

SEQ ID NO: 12 - protein, 11 aa, synthetic construct
ESYLYNADLLM

SEQ ID NO: 13 - protein, 11 aa, synthetic construct
FSLHPLTILWL

SEQ ID NO: 14 - protein, 11 aa, synthetic construct
GTLVPGLKSLV

SEQ ID NO: 15 - protein, 11 aa, synthetic construct
VGAQHIMVSEA

SEQ ID NO: 16 - protein, 11 aa, synthetic construct
THFQSTNKVAL

SEQ ID NO: 17 - protein, 11 aa, synthetic construct
VVLDGSVATPI

SEQ ID NO: 18 - protein, 12 aa, synthetic construct
AALHVACSMALH

SEQ ID NO: 19 - protein, 11 aa, synthetic construct
YFWPFCLASPS

SEQ ID NO: 20 - protein, 11 aa, synthetic construct
PILRTVFGFRW

SEQ ID NO: 21 - protein, 11 aa, synthetic construct
SNVTIVYNSTL

SEQ ID NO: 22 - protein, 11 aa, synthetic construct
PCFSTHLSDIK

SEQ ID NO: 23 - protein, 11 aa, synthetic construct
TKLANFLWMLS

SEQ ID NO: 24 - protein, 11 aa, synthetic construct
CAFTFGYMTFT

SEQ ID NO: 25 - protein, 11 aa, synthetic construct
FFLLVGAQHIM

SEQ ID NO: 26 - protein, 11 aa, synthetic construct
GLTLPLTALVA

SEQ ID NO: 27 - protein, 11 aa, synthetic construct
IFLNCAFTFGY

SEQ ID NO: 28 - protein, 10 aa, synthetic construct
GGPHLMAALH

SEQ ID NO: 29 - protein, 11 aa, synthetic construct
VACSMALHMLT

SEQ ID NO: 30 - protein, 11 aa, synthetic construct
HLVINHIRLLH

SEQ ID NO: 31 - protein, 14 aa, synthetic construct
RLCISQHGLTLPLT

SEQ ID NO: 32 - protein, 11 aa, synthetic construct
AALHVACSMAL

SEQ ID NO: 33 - protein, 11 aa, synthetic construct
ALAANSSSTLC

SEQ ID NO: 34 - protein, 11 aa, synthetic construct
STAGFVHGRYV

SEQ ID NO: 35 - protein, 11 aa, synthetic construct
RSSWCPLLISL

SEQ ID NO: 36 - protein, 11 aa, synthetic construct
LIAVHSSIFSS

SEQ ID NO: 37 - protein, 11 aa, synthetic construct
LWMLSRSSWCP

SEQ ID NO: 38 - protein, 11 aa, synthetic construct
VHGRYVLSSIY

SEQ ID NO: 39 - protein, 11 aa, synthetic construct
FQQWLIAVHSS

SEQ ID NO: 40 - protein, 11 aa, synthetic construct
MAHRLSCKADM

SEQ ID NO: 41 - protein, 11 aa, synthetic construct
IGGMAHRLSCK

SEQ ID NO: 42 - protein, 14 aa, synthetic construct
MAHQCACFHFFLCG

SEQ ID NO: 43 - protein, 11 aa, synthetic construct
GTDWLANRFDW

SEQ ID NO: 44 - protein, 11 aa, synthetic construct
HPLTILWLVFF

SEQ ID NO: 45 - protein, 11 aa, synthetic construct
FWPFCLASPSP

SEQ ID NO: 46 - protein, 11 aa, synthetic construct
HQCACFHFFLC

SEQ ID NO: 47 - protein, 12 aa, synthetic construct
FSLHPLTILWLV

SEQ ID NO: 48 - protein, 11 aa, synthetic construct
MLFTPSQLGSL

SEQ ID NO: 49 - protein, 10 aa, synthetic construct
MKWGPCKAFL

SEQ ID NO: 50 - protein, 11 aa, synthetic construct
FICYLVHSALA

SEQ ID NO: 51 - protein, 11 aa, synthetic construct
CFSLHPLTILW

SEQ ID NO: 52 - protein, 11 aa, synthetic construct
TRPRLPVSWSF

SEQ ID NO: 53 - protein, 11 aa, synthetic construct
CFAVLANASNS

SEQ ID NO: 54 - protein, 11 aa, synthetic construct
ELNGTDWLANR

SEQ ID NO: 55 - protein, 14 aa, synthetic construct
QVPSRKSSQCREAV

SEQ ID NO: 56 - protein, 11 aa, synthetic construct
FLCGFICYLVH

SEQ ID NO: 57 - protein, 11 aa, synthetic construct
REAVGVPQYIT

SEQ ID NO: 58 - protein, 11 aa, synthetic construct
FVHGRYVLSSI

SEQ ID NO: 59 - protein, 11 aa, synthetic construct
LTLCELNGTDW

SEQ ID NO: 60 - protein, 11 aa, synthetic construct
SSIYAVCALAA

SEQ ID NO: 61 - protein, 11 aa, synthetic construct
LWRMMGHAWTP

SEQ ID NO: 62 - protein, 176 aa, synthetic construct
MRIVYLLLPFILLLAQGAAGSSQALGRKSDCFRKSGFCAFLKCPSLTLISGKCSRFYLCCKRIWGEAAAKGLLVLPPIITAAAYSVPDAWYFYYAAYERNNAQLEFGPGPGALTSDEERNNGPGPGGSGVPDNENGPGPRLSSLSVLAKKDVVTEQYRPPKKKLGGPKPPKVGSSG

SEQ ID NO: 63 - protein, 395 aa, synthetic construct
MRIVYLLLPFILLLAQGAAGSSQALGRKSDCFRKSGFCAFLKCPSLTLISGKCSRFYLCCKRIWGEAAAKIPQNAPNGIVFAAYTYIKWPWYVWLAAYGLLVLPPIITAAAYKLGGPKPPKVAAYGRKPVPDAWYFYYAAYERNNAQLEFDDEPKVAAYRFSDGGPDGNFGPGPGRLSSLSVLASGPGPGLLVLPPIITGPGPGITAGDVVTLGPGPGRINHLGITQGPGPGNLTVTDEVIGPGPGLSILKTYIKGPGPGDFDFDDELSKGPGPGFEGSGVPDNENGPGPGVINWGDSALGGPGPGRAAKIIQDQKKGGPGPGRSTAASSAAKKDVVTEQYRPKKSYVYYYQSAFRKKKLGGPKPPKVGSSGKKKATKGKTDAPAKKRSNQGTRD